(19) United States
(12) Patent Application Publication
(10) Pub. No.: US 2022/0053796 A1
(43) Pub. Date: Feb. 24, 2022

(54) COMPOSITION FOR SUPPORTING ANIMAL WITH CANCER

(71) Applicant: MARS, INCORPORATED, McLean, VA (US)

(72) Inventors: Julie Bayle, Aimargues (FR); Delphine Moniot, Aimargues (FR); Vincent Biourge, Aimargues (FR); Yann Queau, Aimargues (FR); Geoffrey Daniel, Aimargues (FR)

(73) Assignee: MARS, INCORPORATED, McLean, VA (US)

(21) Appl. No.: 17/251,673

(22) PCT Filed: Jun. 13, 2019
(86) PCT No.: PCT/US2019/037090

§ 371 Date: Dec. 11, 2020

(30) Foreign Application Priority Data
Jun. 14, 2018 (EP) 18177847.3

(51) Int. Cl. A23K 10/30; A23K 50/42; A23K 50/48; A23K 20/179; A23K 20/174; A23K 20/158; A23K 20/147

(52) U.S. Cl. CPC A23K 10/30; A23K 50/42; A23K 50/48; A23K 20/147; A23K 20/174; A23K 20/158; A23K 20/179

(57) ABSTRACT

The present disclosure relates to a dog food composition comprising from about 10% by weight to about 20% by weight of fat, from about 5% by weight to about 15% by weight of fibers and from about 30% by weight to about 60% by weight proteins, the weight percentages being based on the total weight of dry matter of the composition.

### CROSS REFERENCE TO RELATED APPLICATION

This application is a U.S. National Stage patent application under 35 U.S.C. § 371 of International Patent Application No. PCT/US19/37090, filed on Jun. 13, 2019, which claims the benefit of priority to European Patent Application No. EP18177847.3 filed Jun. 14, 2018, the contents of each of which are hereby incorporated by reference in their entireties, and to each of which priority is claimed.

### FIELD OF THE DISCLOSURE

The present disclosure relates to the field of food compositions for dogs affected with cancer.

### BACKGROUND OF THE DISCLOSURE

The maintenance and improvement of animal health is a constantly ongoing aim in the art. Like their human counterparts, pets that live in developed countries have seen their life expectancy consistently prolonged. Therefore, the global burden of cancers continues to increase largely because of the aging and growing dog population. As an example, the incidence rate of cancers in the dog population is estimated to be from 282.2 to 958 per 100,000 dogs. The most frequent tumors in dogs are mammary tumors in females (70.5% of all cancers), non-Hodgkin's lymphomas (8.4% in females and 20.1% in males) and skin tumors (4% in females and 19.9% in males). Moreover, according to the European Society of Veterinary Oncology 50% of dogs over ten years are going to die from a cancer-related problem.

Chemotherapy is more and more used in animal oncology. Taking advantages of medical advances in human cancer therapy, there are more and more molecules available like vincristine, cyclophosphamide, carboplatin or cisplatin, to treat companion animals. In the veterinary field, anticancer drugs are particularly used in the treatment of tumors derived from hematopoietic tissue (lymphomas, leukemia). For example, the CHOP-based protocol, combining cyclophosphamide, doxorubicin, vincristine and prednisone is currently used in the treatment of numerous lymphoma. Chemotherapeutic agents can be particularly efficient in prolonging the life span of a cancerous animal from a few weeks to several months (the median survival time of dogs treated with the CHOP protocol is 13 months).

Indeed, pet owners, especially owners of companion animals such as dogs, are increasingly opting to treat their pets with chemotherapy to prolong a good Quality Of Life (QOL) as long as possible. Dogs can experience gastrointestinal upset in the form of reduced appetite, nausea, or diarrhea which can reduce their QOL and could result in treatment modifications or even discontinuation of treatment.

An integrative approach for managing a patient with cancer should target the multiple biochemical and physiologic pathways that support tumor development and minimize normal-tissue toxicity. Nutritional intervention is a key component to enhance response to therapy and to improve QOL. Moreover, specific nutrients can be used as powerful tools to reduce toxicity associated with anticancer therapy.

Owners of pets with cancer often inquire about which diets would be the most appropriate to feed to help support the pet during cancer treatment and the internet and other resources describe a number of “cancer diets” (e.g. raw diets, high or moderate protein/low carbohydrate diets, “ketogenic” diets, high fat, omega-3 fatty acid supplementation) purported to improve the outcome in dogs with cancer or to minimize side effects of therapy.

The only existing product aiming at solving this issue consists in a wet product referred as “Prescription Diet™ n/d™ Canine”. It was associated with some modest survival benefits in a selected group of dogs with lymphoma in a clinical trial when used in conjunction with doxorubicin chemotherapy. This product is not usually used or prescribed by veterinarians and is not commonly used in clinical practice. First, as it is a wet product, it presents more constraints in its use as do dry products. It is available only in a canned form which can be inconvenient and cost-prohibitive for some pet owners and it also has an anecdotal reputation for causing gastrointestinal upset. Moreover, this product comprises, as it is commonly admitted by the general knowledge, moderate amount of protein and carbohydrates but it is supplemented with high doses of fish oil. It is generally considered by the person skilled in the art that high level of fat is necessary to this kind of food as it represents a necessary source of energy for the pet that the tumor cannot easily use. It was found that cancer cells are programmed to increase glucose uptake and shift their energy production from mitochondrial oxidative phosphorylation to cytosolic glycolysis, although it is a less efficient pathway; this metabolic hallmark of cancer cells is called the “Warburg effect”. Because of this increase glucose uptake by cancer cells, which is used to fuel their growth and proliferation, it was assumed that a diet for cancer patient should have a moderate level of carbohydrates, in order to put tumor cells in an “unfavorable” situation. Conversely, since many cancer cells synthetize most of the fatty acids they need endogenously (de novo lipogenesis) rather than using circulating lipids, it was assumed that a high fat diet in cancer patient would benefit the host cells rather than the tumor cells.”

There are currently no commercially available diets specifically formulated and clinically validated to support optimal nutritional status and quality of life of pets having cancer, and to help reduce the gastrointestinal side effects of chemotherapy.

### SUMMARY

The present disclosure relates to a pet food composition comprising from about 10% by weight to about 20% by weight of fat, from about 5% by weight to about 15% by weight of fibers and from about 30% by weight to about 60% by weight proteins, the weight percentages being based on the total weight of dry matter of the composition.

In some embodiments, the pet food composition of the present disclosure consists of a wet pet food composition.

In some other embodiments, the pet food composition of the present disclosure consists of a semi-moist pet food composition.

In some further embodiments, the pet food composition of the present disclosure consists of a dry pet food composition.

In some embodiments, the pet food composition of the present disclosure consists of a wet dog food composition.

In some other embodiments, the pet food composition of the present disclosure consists of a semi-moist dog food composition.

In some further embodiments, the pet food composition of the present disclosure consists of a dry dog food composition.

The present disclosure also relates to a dog food composition comprising from about 10% by weight to about 20% by weight of fat, from about 5% by weight to about 15% by weight of fibers and from about 30% by weight to about 50% by weight proteins, the weight percentages being based on the total weight of dry matter of the composition.

In some embodiments, the above pet food composition of the present disclosure consists of a wet dog food composition.

In some other embodiments, the above pet food composition of the present disclosure consists of a semi-moist dog food composition.

In some further embodiments, the above pet food composition of the present disclosure consists of a dry dog food composition.

In some embodiments, the said food composition according to the present disclosure further comprises carbohydrates in an amount ranging from about 15% by weight to about 40% by weight, based on the total weight of dry matter of the composition.

In some embodiments, the said food composition further comprises a source of antioxidants.

In some embodiments, the source of antioxidants comprises one or more vitamins selected in the group consisting of Vitamin C, Vitamin E and carotenoid.

In most preferred embodiments, the said food composition consists of a dry pet food composition and the source of antioxidants comprises Vitamin C wherein Vitamin C is present in the food composition in an amount ranging from about 220 ppm to about 440 ppm on a dry matter basis, and/or Vitamin E wherein Vitamin E is present in the food composition in an amount ranging from about 660 ppm to about 1100 ppm on a dry matter basis and/or carotenoid wherein carotenoid is present in the food composition in an amount ranging from about 2 ppm to about 12 ppm on a dry matter basis.

In other most preferred embodiments, the said food composition consists of a wet pet food composition and the source of antioxidants comprises Vitamin C wherein Vitamin C is present in the food composition in an amount ranging from about 200 to about 600 ppm on a dry matter basis, and/or Vitamin E wherein Vitamin E is present in the food composition in an amount ranging from about 600 ppm to about 2000 ppm on a dry matter basis and/or carotenoid wherein carotenoid is present in the food composition in an amount ranging from about 30 ppm to about 100 ppm on a dry matter basis.

In some embodiments, the said food composition is a dry dog food composition comprising a source of antioxidants, and wherein the source of antioxidants can comprise Vitamin C in an amount ranging from about 200 ppm to about 400 ppm as fed, and/or Vitamin E in an amount ranging from about 600 ppm to about 1000 ppm as fed and/or carotenoid in an amount ranging from about 2 ppm to about 10 ppm as fed.

In some embodiments, the said food composition further comprises a source of curcuminoids.

In most preferred embodiments, the said food composition comprises an amount of curcuminoids ranging from about 250 ppm to about 2000 ppm on a dry matter basis.

In some embodiments, the said food composition further comprises a turmeric extract, such as in an amount ranging from about 300 ppm to about 700 ppm as fed.

In some embodiments, the said food composition further comprises a source of carnosic acid/carnosol.

In some embodiments, the said food composition further comprises a rosemary extract, as a source of carnosic acid and carnosol. As for the other ingredients of the pet food composition, the amount of rosemary extract which is present in the pet food composition can vary depending on the content of the said extract in carnosic acid and carnosol.

In some embodiments, the said food further comprises a rosemary extract, which rosemary extract can be present in an amount ranging from about 50 ppm to about 120 ppm as fed.

In most preferred embodiments, the said food composition comprises an amount of carnosic acid and carnosol in an amount ranging from about 20 ppm to about 90 ppm on a dry matter basis.

In some embodiments, the said food composition further comprises a source of piperine.

In some embodiments, the said food composition is a dry dog food and further comprises a source of piperine.

In some embodiments, the source of piperine can consist of a pepper extract.

In some embodiments of the food composition according to the present disclosure, the source of piperine consists of a pepper extract and the pepper extract is present in the composition in an amount ranging from about 15 ppm to about 35 ppm as fed.

In most preferred embodiments, the food composition according to the present disclosure comprises an amount of piperine ranging from 14 ppm to 60 ppm on a dry matter basis.

In some embodiments, the food composition according to the present disclosure, the source of piperidine consists of a pepper extract.

The pepper extract, depending of the content of the said pepper extract in piperine, is most preferably present in the composition in an amount suitable for obtaining a final content in the food composition ranging from 14 ppm to 60 ppm on a dry matter basis.

In embodiments wherein the said pet food composition is a dry dog food composition which comprises a source of piperine under the form of a pepper extract, pepper extract is present in the dry dog food composition in an amount ranging from 15 to 35 ppm as fed

In some embodiments, the said food composition comprises a turmeric extract and/or a rosemary extract, and/or a pepper extract, and/or a green tea extract, and/or a pomegranate extract. In some of these embodiments, the said food composition comprises a source of curcuminoids and of a rosemary extract, and optionally also a source of piperine. In some embodiments, the said food composition a dry dog food composition.

This disclosure also relates to a food composition as defined throughout the present specification, for use in supporting dogs affected with cancer and undergoing chemotherapy.

### DESCRIPTION OF THE FIGURES

FIG. 1 illustrates fecal scores with time of animals fed with moderate fat nutritional composition.

Ordinate: mean value of weekly fecal score.

Abscissa: (i) left panel: test group of animals; (ii) right panel: control group of animals. In each of left panel and right panel, and from left to right of each panel: time period after the starting of feeding the animals with the composition (left panel) or with the convention composition (right panel): (i) week 1, (ii) week 2, (iii) week 3, (iv) week 4, (v) week 5, (vi) week 6, (vii) week 7, (viii) week 8.

FIG. 2 illustrates the global QOL score values with time of animals fed with moderate fat nutritional composition.

Ordinate: percent of case

Abscissa: (1) each group of paired bars, from left to right: (i) animals fed with moderate fat nutritional composition, (ii) animals fed with conventional nutritional composition. (2) groups of paired bars, from left to right: (i) baseline, (ii) week 2, (iii) week 4, (iv) week 6, (v) week 8. In each bar, colored sections, from top to bottom: (i) Poor, (ii) Fair, (iii) Good, (iv) Very good, (v) Excellent. In each section the number represent the number of animals.

FIG. 3: Anti-proliferative effects of plant extracts on various dog tumor cell lines.

FIG. 3A illustrates the anti-proliferative activity of a pomegranate 40% ellagic acid extract (POE40®) on various dog tumor cell lines. Ordinate, from the upper part to the lower part of the figure: BACA, BR, C2, CF33.MT, CF41.Mg, CLBL-1, D17, HMPOS, K9, Abscissa: final concentration of extract in the cell culture, as expressed in μg/ml.

FIG. 3B illustrates the anti-proliferative activity of a green tea extract (Naturex) on various dog tumor cell lines. Ordinate, from the upper part to the lower part of the figure: BACA, BR, C2, CF33.MT, CF41.Mg, CLBL-1, D17, HMPOS, K9, Abscissa: final concentration of extract in the cell culture, as expressed in μg/ml.

FIG. 3C illustrates the anti-proliferative activity of a black pepper extract (VETPERINE®) on various dog tumor cell lines. Ordinate, from the upper part to the lower part of the figure: BACA, BR, C2, CF33.MT, CF41.Mg, CLBL-1, D17, HMPOS, K9, Abscissa: final concentration of extract in the cell culture, as expressed in μg/ml.

FIG. 3D illustrates the anti-proliferative activity of a rosemary Extract (INOLENS70®) on various dog tumor cell lines. Ordinate, from the upper part to the lower part of the figure: BACA, BR, C2, CF33.MT, CF41.Mg, CLBL-1, D17, HMPOS, K9, Abscissa: final concentration of extract in the cell culture, as expressed in μg/ml.

FIG. 3E illustrates the anti-proliferative activity of a turmeric roots (Naturex) extract on various dog tumor cell lines. Ordinate, from the upper part to the lower part of the figure: BACA, BR, C2, CF33.MT, CF41.Mg, CLBL-1, D17, HMPOS, K9, Abscissa: final concentration of extract in the cell culture, as expressed in μg/ml.

FIG. 3F illustrates the anti-proliferative activity of a pomegranate 40% punicosides extract (P40P®) on various dog tumor cell lines. Ordinate, from the upper part to the lower part of the figure: BACA, BR, C2, CF33.MT, CF41.Mg, CLBL-1, D17, HMPOS, K9. Abscissa: final concentration of extract in the cell culture, as expressed in μg/ml.

FIG. 4 illustrates anti-proliferative effects of combinations of two plant extracts on various dog tumor cell lines.

FIG. 4A illustrates the anti-proliferative effect of the combination of a rosemary extract and of a turmeric extract on the proliferation of the C2 cancer cell line. Curves: (i) dashed line: turmeric extract, (ii) dotted line: rosemary extract at 70% w/w carnosic acid; (iii) continuous line: combination of turmeric extract and rosemary extract at 70% w/w carnosic acid. Ordinate: Percent proliferating cells as compared to the control culture in the absence of these extracts. Abscissa: final concentration of the combination of extracts in the cell culture, as expressed in μg/ml.

FIG. 4B illustrates the anti-proliferative effect of the combination of a rosemary extract and of a turmeric extract on the proliferation of the CMT-12 cancer cell line. Curves: (i) dashed line: turmeric extract, (ii) dotted line: rosemary extract at 70% w/w carnosic acid; (iii) continuous line: combination of turmeric extract and rosemary extract at 70% w/w carnosic acid. Ordinate: Percent proliferating cells as compared to the control culture in the absence of these extracts. Abscissa: final concentration of the combination of extracts in the cell culture, as expressed in μg/ml.

FIG. 4C illustrates the anti-proliferative effect of the combination of a rosemary extract and of a turmeric extract on the proliferation of the D17 cancer cell line. Curves: (i) dashed line: turmeric extract, (ii) dotted line: rosemary extract at 70% w/w carnosic acid; (iii) continuous line: combination of turmeric extract and rosemary extract at 70% w/w carnosic acid. Ordinate: Percent proliferating cells as compared to the control culture in the absence of these extracts. Abscissa: final concentration of the combination of extracts in the cell culture, as expressed in μg/ml.

FIG. 5 illustrates the anti-proliferative activity of distinct plant extracts from different sourcing on various dog tumor cell lines.

FIG. 5A illustrates the anti-proliferative activity of the following combination of extracts: (i) turmeric extract (Naturex) and rosemary extract INOLENS70®, (ii) turmeric extract (Naturex) and rosemary extract INOLENS50®, (iii) turmeric extract BCM-95® and rosemary extract INOLENS70® and (iv) turmeric extract BCM-95® and rosemary extract INOLENS50®, on the dog tumor cell line C2. Ordinate: percent of viable cells as compared to the control culture performed in the absence of the said combinations of plant extracts. Abscissa: final concentration of the combination of extracts in the cell culture, as expressed in μg/ml.

FIG. 5B illustrates the anti-proliferative activity of the following combination of extracts: (i) turmeric extract and rosemary extract INOLENS70®, (ii) turmeric extract and rosemary extract INOLENS50®, (iii) turmeric extract BCM-95® and rosemary extract INOLENS70® and (iv) turmeric extract BCM-95® and rosemary extract INOLENS50®, on the dog tumor cell line CMT-12. Ordinate: percent of viable cells as compared to the control culture performed in the absence of the said combinations of plant extracts. Abscissa: final concentration of the combination of extracts in the cell culture, as expressed in μg/ml.

FIG. 5C illustrates the anti-proliferative activity of the following combination of extracts: (i) turmeric extract and rosemary extract INOLENS70®, (ii) turmeric extract and rosemary extract INOLENS50®, (iii) turmeric extract BCM-95® and rosemary extract INOLENS70® and (iv) turmeric extract BCM-95® and rosemary extract INOLENS50®, on the dog tumor cell line D17. Ordinate: percent of viable cells as compared to the control culture performed in the absence of the said combinations of plant extracts. Abscissa: final concentration of the combination of extracts in the cell culture, as expressed in μg/ml.

FIG. 6 illustrates the anti-proliferative activity of the natural extracts in a soft agar colony formation assay. Ordinate: percent viable cells as compared to the control culture performed in the absence of extracts. Bars in the abscissa, from left to right: (i) Control (DMSO), (ii) 0.4 μg/ml curcumin extract (Turmeric extract from Naturex), (iii) 0.8 μg/ml rosemary INOLENS70®, (iv) 6.25 μg/ml VETPERINE®, (v) 0.4 μg/ml Curcumin extract (Naturex) and 0.8 μg/ml rosemary INOLENS70®, and (vi) 0.4 μg/ml Curcumin extract (Naturex), 0.8 μg/ml rosemary INOLENS70® and 6.25 μg/ml VETPERINE®.

FIG. 7 illustrates the cytotoxic activity of various plant extracts on canine cells. Ordinate: percent of viable cells as compared to the control culture without the said plant extract. Abscissa: (1) group of bars, from left to right, the following dog tumor cell lines: (i) CDF, (ii) C2, (iii) CMT-12, (iv) D17; (2) In each group of bars, from left to right: (i) Control (DMSO), (ii) 6.3 μg/ml turmeric extract (Naturex), (iii) 6.3 μg/ml rosemary extract INOLENS70®, (iv) 3.1 μg/ml turmeric extract (Naturex), 3.1 μg/ml rosemary extract INOLENS70®

FIG. 8 illustrates mechanisms of the anti-proliferative activity and cytotoxic effect of plant extracts.

FIGS. 8A to 8D consist of the results of flow cytometry assays wherein each quadrant represents the number of events corresponding to (i) lower left quadrant: cells assessed as being alive, (ii) lower right quadrant cells assessed as being early apoptotic, (iii) upper right quadrant: cells assessed as being late apoptotic/necrotic. Ordinate: fluorescence signal of 7-AAD-expressing cells, as expressed in Arbitrary Units. Abscissa: fluorescence signal of Annexin-V-positive cells, as expressed in Arbitrary Units.

FIG. 8E illustrates the percentage of early apoptotic cells (lower right quadrant of Annexin V positive and 7-AAD negative cells) are represented as mean f standard deviation 3 independent replicates. Within each cell line, means with different letters are significantly different from each other (p<0.05). Ordinate: percent apoptotic cells. Abscissa: (1) group of bars, from left to right, the following dog tumor cell lines: C2, CMT-12 and D17. (2) for each group of bars, from left to right: (i) Control (DMSO), (ii) 6.3 μg/ml turmeric extract (Naturex), (iii) 6.3 μg/ml rosemary extract INOLENS70@, (iv) 3.1 μg/ml turmeric extract, 3.1 μg/ml rosemary extract INOLENS70®.

FIG. 9 illustrates the apoptotic activity of plant extracts. Ordinate: fold change of activated caspase 3/7. Abscissa: (1) group of bars, from left to right, the following dog tumor cell lines: C2, CMT-12 and D17. (2) for each group of bars, from left to right: (i) Control (DMSO), (ii) 6.3 μg/ml turmeric extract (Naturex), (iii) 6.3 μg/ml rosemary extract INOLENS70®, (iv) 3.1 μg/ml turmeric extract (Naturex), 3.1 μg/ml rosemary extract INOLENS70®.

FIG. 10 illustrates the antioxidant activity of plant extracts. Ordinate: fold change in production of Reactive Oxygen Species (ROS). Abscissa: (1) group of bars, from left to right, the following dog tumor cell lines: C2, CMT-12 and D17. (2) for each group of bars, from left to right: (i) Control (DMSO), (ii) 6.3 μg/ml turmeric extract (Naturex), (iii) 6.3 μg/ml rosemary extract INOLENS70®, (iv) 3.1 μg/ml turmeric extract (Naturex), 3.1 μg/ml rosemary extract INOLENS70®.

FIG. 11 illustrates the SAPK/JNK pathway activation by plant extracts.

FIG. 11A represents the results of a Western blotting experiment performed with the C2 cell line. FIG. 11B represents the results of a Western blotting experiment performed with the CMT-12 cell line.

FIGS. 11A and 11B, lanes from left to right: (i) Control (DMSO) after 12 h incubation, (ii) Control (DMSO) after 24 h incubation, (iii) turmeric extract (Naturex) at 6.3 μg/ml after 12 h incubation, (iv) turmeric extract (Naturex) at 6.3 μg/ml after 24 h incubation, (v) rosemary extract (INOLENS70®) at 6.3 μg/ml after 12 h incubation, (vi) rosemary extract (INOLENS70®) at 6.3 μg/ml after 24 h incubation, (vii) 3.1 μg/ml of a combination of turmeric extract with rosemary extract after 12 h incubation, (viii) 3.1 μg/ml of a combination of turmeric extract with rosemary extract after 24 h incubation.

FIG. 12 illustrates accumulation of curcumin by cells treated by rosemary extract.

FIG. 12A: effect on C2 cell line. FIG. 12B: effect on CMT-12 cell line. FIG. 12C: effect on D17 cell line.

In FIGS. 12A, 12B and 12C. Ordinate: fold change in curcumin, as compared to the control culture without rosemary extract. Abscissa, bars from left to right: (i) Control (DMSO), (ii) 3.1 μg/ml turmeric extract (Naturex), (iii) 3.1 μg/ml rosemary extract INOLENS70@, (iv). 3.1 μg/ml turmeric extract (Naturex) and 3.1 μg/ml rosemary extract INOLENS70®

FIG. 13 illustrates the treatment schedule of Example 3.

### DETAILED DESCRIPTION

The present disclosure aims at making available a nutritional product for dogs diagnosed with cancer (i) undergoing chemotherapy and/or (ii) after a treatment period by chemotherapy (daily consumption). This food composition should be capable of maintaining good QOL, limiting side effects chemotherapy (e.g. helping dogs fighting their cancer, through “support to treatment”) and maintaining a good nutritional status (bodyweight maintenance, good digestive health).

This goal has been reached by providing a nutritional composition with a high amount of protein and a moderate amount of carbohydrate and a moderate amount of fat. As it is shown in the examples herein, such a nutritional composition, when provided to dogs affected with cancer, especially to dogs undergoing an anticancer chemotherapeutic treatment, is well tolerated and substantially reduces the signs of illness and significantly improves their quality of life. Further, the improvement of the quality of life of cancer dogs fed with the high protein/moderate carbohydrate and fat disclosed herein readily occurs after a short period of time after the beginning of this food regimen, typically occurs only four weeks after the beginning of this food regimen.

The present disclosure relates to a food composition with a high amount of protein and moderate amount of carbohydrate and fat. Such a composition can be used for supporting an animal with cancer and more particularly an animal with cancer ongoing treatment such as chemotherapy. The food composition can be also used in a method of supporting an animal with a cancer undergoing therapy, such as a chemotherapy. The composition can also be used in a method for the treatment of cancer.

The present disclosure relates to a food composition comprising from about 10% by weight to about 20% by weight of fat, from about 5% by weight to about 15% by weight of fibers and from about 30% by weight to about 60% by weight proteins, the weight percentages being based on the total weight of dry matter of the composition.

In some embodiments, the food composition of the present disclosure consists of a wet pet food composition, such as a wet dog food composition.

In some other embodiments, the food composition of the present disclosure consists of a semi-moist pet food composition, such as a semi-moist dog food composition.

In some further embodiments, the food composition of the present disclosure consists of a dry pet food composition, such as a dry dog food composition.

As used herein, “about” or “approximately” means within an acceptable error range for the particular value as determined by one of ordinary skill in the art, which will depend in part on how the value is measured or determined, i.e., the limitations of the measurement system. For example, “about” can mean within three or more than three standard deviations, per the practice in the art. Alternatively, “about” can mean a range of up to 20%, preferably up to 10%, more preferably up to 5%, and more preferably still up to 1% of a given value. Also, particularly with respect to systems or processes, the term can mean within an order of magnitude, preferably within five-fold, and more preferably within two-fold, of a value.

As used herein, the terms “dry pet food”, “dry food”, “wet pet food”, “wet food”, “semi-moist pet food” and “semi-moist food” designate a nutritionally complete pet food composition encompassing any product which a pet animal consumes in its diet. The pet food composition is preferably a cooked product. It can incorporate meat or animal derived material (such as beef, chicken, turkey, lamb, fish, blood plasma, marrow bone etc. or one or more thereof). The pet food composition alternatively can be meat free (preferably including a meat substitute such as soya, maize gluten or a soya product) in order to provide a protein source.

As used herein, a “dry” pet food composition has a moisture content of 15% or less, such as a moisture content ranging from 1% to 15%.

As used herein, a “semi-moist” pet food composition has a moisture content ranging from more than 15% to 50%.

As used herein, a “wet” pet food composition has a moisture content of 90% or less, such as a moisture content ranging from more than 50% to 90%.

In some of its aspects, the present disclosure relates to a food composition useful for dogs with cancer, comprising fat, fibers and proteins, characterized in that it comprises fat in an amount ranging from about 10% to about 20% on a dry matter basis, fibers in an amount ranging from about 5% to about 15% on a dry matter basis and proteins in an amount ranging from about 30% to about 50% on a dry matter basis.

The present disclosure relates to a food composition comprising fat, fibers and proteins, and wherein fat is comprised in an amount ranging from about 10% by weight to about 20% by weight, fibers are comprised in an amount ranging from about 5% by weight to about 15% by weight and proteins are comprised in an amount ranging from about 30% by weight to about 50% by weight, based on the total weight of dry matter of the composition.

The present disclosure relates to a food composition comprising from about 10% by weight to about 20% by weight of fat, from about 5% by weight to about 15% by weight of fibers and from about 30% by weight to about 50% by weight proteins, the weight percentages being based on the total weight of dry matter of the composition.

The expressions “dry matter (DM) basis” must be interpreted as a method of expressing the concentration of a nutrient or a component in a feed by expressing its concentration relative to its dry matter content (the concentration remaining once the moisture has been taken out). At the opposite, the expression “as fed” must be interpreted as a method of expressing the concentration of a nutrient or a component in a feed by expressing its concentration in the state it is fed, which includes moisture.

As previously mentioned, the food composition of present disclosure can consist of a dry pet food composition, or of a semi-moist pet food composition, or of a wet pet food composition.

The respective amounts of fat, fibers, protein and carbohydrates in a pet food composition described herein are expressed in reference of the total weigh of dry matter of the said composition, thus irrespective of the moisture content of the said pet food composition.

Other ingredients that can be comprised in the food composition described throughout the present specification can be expressed in “ppm” units (also termed “parts per million”), which is another conventional way of specifying an amount of a substance comprised in a composition, including when comprised in a pet food composition.

Throughout the present specification, an amount of a given substance that is expressed in “ppm” units, means (i) an amount in the composition “as fed” or (ii) an amount in ppm on “a dry matter basis”, as it will be specified.

When an amount of a given substance is expressed herein in ppm “as fed”, the amount value in ppm can change according to the moisture content of the pet food composition. Illustratively, a pet food composition having 50% moisture content and comprising an amount of 100 ppm “as fed” of a given substance has the same amount of the said given substance if expressed in a dry matter basis, than a pet food composition having 30% moisture content and comprising 140 ppm “as fed” of the said given substance, i.e. in both cases a pet food composition comprising 200 ppm of the said given substance on a dry matter basis

As it is usual in the art, an amount of a given substance comprised in a pet food composition as expressed in “ppm as fed” is easily converted in the amount of the said given substance as expressed in reference to the total dry matter of the pet food composition using the formula below:

Y mg/100 g DM=X ppm“as fed”)*100/(% dry matter), wherein
Y is the amount in mg of the said given substance per 100 g of dry matter (“DM”) contained in the pet food composition.
X is the amount in ppm of the said given substance in the pet food composition,
% dry matter is the percentage of dry matter in the pet food composition,

It can also be used the other formula below:

(% X“as fed”)*100/(% dry matter)=Y g/100 g DM, wherein
% X is the amount in weight percentage of the said given substance in the “ready to eat” pet food composition,
% dry matter is the percentage of dry matter in the “ready to eat” pet food composition,
Y is the amount in g of the said given substance per 100 g of dry matter (“DM”) contained in the “ready to use” pet food composition.

For the purpose of the present disclosure, unless otherwise indicated, the percentage of a substance comprised in the dry dog food composition consists of a percentage by weight based on the total weight of the dry matter of the said composition.

A first aspect of a food composition as described herein, consists of the moderate level of fat comprised therein. As mentioned above, it is commonly admitted by the scientific community that a high level of fat should present a real interest in food for animals undergoing cancer treatment as it represents a source of energy which is not easy to use for cancer cells. A particular innovative aspect of the present disclosure is that, contrary to this prejudice, it is described for the first time a composition comprising a relatively moderate level of fat. Inventors have defined an optimal fat content that shall be comprised in a food composition for use in dogs affected with cancer, and especially for cancer dogs undergoing an anti-cancer treatment, e.g. a cancer treatment by chemotherapy. The inventors have determined herein that the content of fat shall not be too low so that the resulting dog food composition possesses at least minimal requirements in the calorie content. However, the inventors have also determined herein that the content of fat shall not be too high so that the resulting dog food composition be well-tolerated by the cancer sick animals. It is well known that cancer treatments of sick animals cause alterations in the functioning of the digestive system. Therefore, the inventors have taken care in designing a composition which is well-tolerated by cancer dogs, especially which is well tolerated by cancer dogs undergoing a cancer treatment.

In a preferred embodiment, a dry dog food composition as described herein comprises fat in an amount ranging from 10% to 20% by weight, preferably ranging from 12% to 16% by weight and still more preferably in an amount of about 13% by weight, on a dry matter basis, i.e. based on the total weight of dry matter of the composition.

The expressions “fat”, or “source of fat” as used in the present specification comprises any food-acceptable fat (s) and/or oil (s) irrespective of their consistency at room temperature, i.e. irrespective whether said “source of fat” is present in essentially fluid form or in essentially solid from. The composition according to the present disclosure can comprise fat of animal and/or vegetable origin. Fat can be supplied by any of a variety of sources known by those skilled in the art. Plant fat sources include, without limitation, wheat, sunflower, safflower, rapeseed, olive, borage, flaxseed, peanuts, blackcurrant seed, cottonseed, wheat, germ, corn germ as well as oils derived from these and other plant fat sources. Animal sources include, for example and without limitation, chicken fat, turkey fat, beef fat, duck fat, pork fat, lamb fat, etc., fish oil or any meat, meat by-products, seafood, dairy, eggs, etc. Fat content of foods can be determined by any number of methods known by those of skill in the art.

A second particular aspect of a food composition as described herein consists of the level of fibers comprised therein.

The expression “fibers” is similar to “dietary fibers” and shall be interpreted for the purpose of the present disclosure as Total Fibers, meaning that it includes soluble fibers and insoluble fibers. Soluble fiber (also referred as fermentable fibers) can be defined as being resistant to digestion and absorption in the small intestine and undergo complete or partial fermentation in the large intestine. As non-limitative example of soluble fibers, it can be mentioned beet pulp, guar gum, chicory root, psyllium, pectin, blueberry, cranberry, squash, apples, oats, beans, citrus, barley, or peas. A preferred soluble fiber is chicory pulp. Soluble fibers are considered as having a prebiotic effect by providing short chain fatty acids as a source of energy to colonocytes. By opposition, insoluble fiber (also referred as non-fermentable fibers) can be defined as non-starch polysaccharides that are resistant to digestion and absorption in the small intestine, and resistant to fermentation in the large intestine. As non-limitative example of insoluble fibers, it can be mentioned cellulose, whole wheat products, wheat oat, corn bran, flax seed, grapes, celery, green beans, cauliflower, potato skins, fruit skins, vegetable skins, peanut hulls, and soy fiber. A preferred insoluble fiber is cellulose. Insoluble fibers are considered as useful for transit and ballast effect. In a preferred embodiment, the composition of the present disclosure comprises fibers in an amount from 5 to 15%, preferably from 7 to 10% and still more preferably around 8.9%, on a dry matter basis.

In another embodiment, a food composition as described herein can also comprise Psyllium for improving digestive health as Psyllium will give consistency to liquid feces and soften dry feces.

Preferentially, a dry dog food composition as disclosed herein comprises an amount of Psyllium ranging from 0.2% by weight to 1% by weight, more preferentially in an amount of about 0.5% by weight, on a dry matter basis, i.e. based on the total weight of dry matter of the composition.

A third particular aspect of a food composition disclosed herein consists of the level of proteins comprised therein. The protein level shall be high so as to ensure maintenance of lean body mass.

A food composition according to the present disclosure can contain one or more distinct proteins. Generally, a food composition as described herein comprises a plurality of proteins that are contained in a protein source which is used in the manufacture process. In some embodiments a protein comprised in a food composition is in a native form. In some other embodiments a protein can be present in an at least partially hydrolysed form, which encompasses a protein which is almost completely hydrolyzed. A food composition according to the present disclosure can incorporate proteins under the form of meat or animal derived material (such as beef, chicken, turkey, lamb, fish, blood plasma, marrow bone etc. or one or more thereof). In some other embodiments, a food composition as described herein can be meat-free and preferably comprises a meat substitute protein source such as soya, maize gluten or any other protein-containing soya product in order to provide a protein source. A food composition as disclosed herein can comprise additional protein sources such as soya protein concentrate, milk proteins, gluten etc.

In some embodiments, a food composition as described herein further comprises carbohydrates in an amount ranging from about 15% by weight to about 40% by weight on a dry matter basis, i.e. based on the total weight of dry matter of the composition.

The expression “carbohydrate” as used herein encompasses polysaccharides and sugars that are metabolized for energy when hydrolyzed in the body. The carbohydrate content of foods can be determined by any number of methods known by those of skill in the art. However, in the present specification, and unless the contrary is clearly specified, the carbohydrate percentage is calculated as nitrogen free extract (“NFE”), which can be calculated as follows:

NFE=100%−moisture %−protein %−fat %−ash %−crude fiber %.

Carbohydrate can be supplied under the form of any of a variety of carbohydrate sources known by those skilled in the art, including oat fiber, cellulose, peanut hulls, beet pulp, parboiled rice, corn starch, corn gluten meal, and any combination of those sources. Grains supplying carbohydrate include, but are not limited to, wheat, corn, barley, and rice.

In preferred embodiments of a food according to the present disclosure, the moisture content of a dry pet food composition according to the present disclosure ranges from 1% by weight to 15% by weight, advantageously ranges from 5% by weight to 12% by weight, and more preferentially ranges from 8% by weight to 10% by weight on a dry matter basis, i.e. based on the total weight of dry matter of the composition. In a most preferred embodiment, the moisture content is of about 9.5% by weight, on a dry matter basis, i.e. based on the total weight of dry matter of the composition.

Further Ingredients

In some embodiments, the food composition according to the present disclosure further comprises a source of antioxidants.

The expression “antioxidant” means a substance or a component that is capable of reacting with free radicals and neutralizing them. Illustrative examples of such substances include, without limitation, carotenoids, including beta-carotene, lycopene and lutein, selenium, coenzyme Q10 (ubiquinone), tocotrienols, soy isoflavones, S-adenosylmethionine, glutathione, taurine, N-acetylcysteine, vitamin E, vitamin C, lipoic acid and L-carnitine.

In a preferred embodiment, the food composition comprises Vitamin C.

In a preferred embodiment, the food composition comprises Vitamin E.

In a preferred embodiment, the food composition comprises Carotenoids. Preferred carotenoids are lutein and beta-carotene.

In a preferred embodiment, the food composition further comprises lutein.

In some embodiments, the food composition comprises a combination of such antioxidants, such as a combination of Vitamin C and/or Vitamin E and/or Carotenoids and/or taurine. In some preferred embodiments, a food composition comprises a combination of Vitamin C and Vitamin E and Carotenoids.

In some preferred embodiments, a food composition according to the present disclosure further comprises a source of antioxidants comprising Vitamin C.

In most preferred embodiments, a food composition according to the present disclosure comprises Vitamin C in an amount ranging from 200 ppm to 600 ppm on a dry matter basis.

In some most preferred embodiments, a food composition according to the present disclosure is a dry pet food composition and comprises a source of antioxidants comprising Vitamin C, and wherein Vitamin C is present in an amount ranging from 220 ppm to 440 ppm on a dry matter basis.

In some most preferred embodiments, a food composition according to the present disclosure is a wet pet food composition and further comprises a source of antioxidants comprising Vitamin C, and wherein Vitamin C is present in an amount ranging from 200 ppm to 600 ppm on a dry matter basis.

In some preferred embodiments, a food composition according to the present disclosure is a dry pet food composition and further comprises a source of antioxidants comprising Vitamin E and wherein the final amount of Vitamin E in the food composition is in an amount ranging from 660 ppm to 1100 ppm on a dry matter basis.

In some preferred embodiments, a food composition according to the present disclosure is a wet pet food composition and further comprises a source of antioxidants comprising Vitamin E and wherein the final amount of Vitamin E in the food composition is in an amount ranging from 600 ppm to 2000 ppm, which encompasses from 800 ppm to 1300 ppm on a dry matter basis.

In some preferred embodiments, a food composition according to the present disclosure is a dry pet food composition and further comprises a source of antioxidants comprising carotenoids.

In most preferred embodiments of a dry pet food according to the present disclosure, carotenoids are present in the food composition in an amount ranging from 2 ppm to 12 ppm on a dry matter basis.

In some other preferred embodiments, a food composition according to the present disclosure is a wet pet food composition and further comprises a source of antioxidants comprising carotenoids.

In some other preferred embodiments, a food composition according to the present disclosure is a wet pet food composition and further comprises carotenoids and wherein carotenoids are present in the said wet pet food in an amount ranging from 2 ppm to 100 ppm, which encompasses from 30 ppm to 100 ppm on a dry matter basis.

As a non-limitative embodiment, it can be used a combination of antioxydants called “CELT cocktail” which comprises Vitamin C (about 300 ppm), Vitamin E (about 800 ppm), Lutein (about 5pp), Taurine (about 0.625 g/MCal)+Vitamin D3 (about 1000 IU/kg).

In another embodiment, the food composition according to the present disclosure can also comprise sodium Butyrate for prebiotic affect as a short chain fatty acid and source of energy for colonocytes. Preferentially, the food composition comprises from 0.2% by weight to 1% by weight of Butyrate sodium, more preferentially about 0.5% by weight of Butyrate sodium on a dry matter basis, i.e. based on the total weight of dry matter of the composition.

In the same goal as Butyrate sodium above, the food composition according to the present disclosure can also comprise Zeolite to improve fecal consistency. Preferentially, the food composition comprises from 0.5% by weight to 1.5% by weight of Zeolite, more preferentially about 1% by weight on a dry matter basis, i.e. based on the total weight of dry matter of the composition.

In some preferred embodiments, the food composition as described herein can also comprise Arginine as a supplement to enhance specific immunity. Preferentially, the food composition comprises from 1% by weight to 4% by weight of Arginine, more preferentially from 2% by weight to 3% by weight and still more preferentially about 2.7% by weight on a dry matter basis, i.e. based on the total weight of dry matter of the composition.

In another embodiment, the food composition can also comprise EPA/DHA to improve metabolic status and generate an anti-inflammatory effect. Preferentially, the food composition comprises from 0.4% by weight to 0.8% by weight of EPA/DHA, more preferentially about 0.6% by weight on a dry matter basis, i.e. based on the total weight of dry matter of the composition.

As it is shown in the examples herein, it can be desirable that a food composition according to the present disclosure further comprises one or more extracts endowed with anti-cancer activity, which encompass substances that are cytotoxic against cancer cells, including substances having an anti-proliferative and/or a pro-apoptotic effect against cancer cells, As it is also shown in the examples herein, it can be desirable that a dry dog food composition according to the present disclosure further comprises a combination of two or more substances endowed with anti-cancer activity, and especially a combination of two or more substances endowed with anti-cancer activity, for which a synergistic anti-cancer activity has been determined. Combinations of substances endowed with anti-cancer activity encompass combinations of two substances endowed with anti-cancer activity as well as combinations of three substances endowed with anti-cancer activity, especially those having a synergistic anti-cancer activity.

Another particular and preferred embodiment of a food composition disclosed herein, is the inclusion of curcuminoids.

Thus, in some embodiments, a food composition as described herein further comprises a source of curcuminoids.

Curcuminoids include curcumin, demethoxycurcumin, bis-methoxycurcumin and/or tetrahydrocurcumin. Curcuminoids are natural phenols that are present, in particular, in the Indian spice turmeric. Turmeric is derived from the roots of the plant Curcuma longa. Curcuminoids have also been found in roots of other species in the plant family Zingiberaceae of the Curcuma genus. In particular, turmeric contains 60-80% curcumin, 15-30% demethoxycurcumin and 2-6% bis-demethoxycurcumin. The curcuminoid in the composition of the present disclosure can be of any format, including a powder or lipid extract.

In most preferred embodiments, a food composition according to the present disclosure, curcuminoids are present in an amount ranging from about 250 ppm to about 2000 ppm on a dry matter basis.

In a preferred embodiment, the source of curcuminoids consists of a turmeric extract (Curcuma Longa). As non-limitative examples, it can be mentioned (i) the Turmeric extract BCM-95@commercialized by Arjuna comprising 86% wt/wt curcuminoids, (ii) the Turmeric extract commercialized by Naturex comprising 85% wt/wt curcuminoids and (iii) the Turmeric extract commercialized by Indena/Meriva comprising 20% wt/wt curcuminoids. Any other sourcing known by the person skilled in the art can also be used. According to such a preferred aspect, a food composition as described herein further comprises a turmeric extract. Other available sources for curcuminoids can also be selected from liposomal curcumin, curcumin nanoparticles, curcumin phospholipid complex, structural analogues of curcumin (e.g EF-24) demethoxycurcumin, bisdemethoxycurcumin, tetrahydrocurcumin, and commercial/DM, any formulation designed to enhance curcumin bioavailability.

As it is readily understood by the skilled artisan, the amount of turmeric extract which is comprised in a pet food composition according to the present disclosure depending on the concentration Illustratively, a pet food composition comprising about 1000 ppm curcuminoids can be selected among (i) a pet food composition comprising about 1163 ppm of a turmeric extract having 86% wt/wt curcuminoids, (ii) a pet food composition comprising about 1176 ppm of a turmeric extract having 85% wt/wt curcuminoids or (iii) a pet food composition comprising about 5000 ppm of a turmeric extract having 20% wt/wt curcuminoids.

In embodiments wherein the food composition is a dry dog food composition, the said turmeric extract can be present in the dry food composition in an amount ranging from 300 ppm to 700 ppm as fed, preferentially from 400 ppm to 600 ppm as fed, and still more preferably in an amount of 500 ppm as fed

The said turmeric extract can be present in a dry dog food composition in an amount ranging from 300 ppm to 700 ppm as fed, preferentially from 400 ppm to 700 ppm as fed, and still more preferably in an amount of 500 ppm as fed,

In yet another preferred embodiment wherein the food composition is a dry dog food composition, the pet food composition can comprise the said turmeric extract present in an amount ranging from about 300 ppm to about 700 ppm.

In the most preferred embodiments, a food composition according to the present disclosure, curcuminoids are present in an amount ranging from about 250 ppm to about 2000 ppm on a dry matter basis.

According to some other embodiments, a food composition as described herein can comprise carnosic acid/carnosol. In some preferred aspect, a food composition as disclosed herein further comprises a source of carnosic acid/carnosol.

In a preferred embodiment, the source of carnosic acid/carnosol consists of a rosemary extract (Rosmarinus oficinalis). As non-limitative example, it can be mentioned the Rosemary extract INOLENS50® or INOLENS70® from Vitiva. Any other sourcing known by the person skilled in the art can also be used. According to such a preferred aspect, a pet food composition as described herein further comprises a rosemary extract. The said rosemary extract can be present in the said food composition in an amount ranging from about 50 ppm to about 120 ppm as fed, preferentially from about 70 ppm to about 100 ppm as fed, and still more preferably in an amount of about 90 ppm on a dry matter basis. In yet another preferred embodiment, the food composition the rosemary extract is present in an amount ranging from about 50 ppm to about 120 ppm as fed. The said rosemary extract can be present in a dry pet food composition having about 10% moisture content in an amount ranging from 55 ppm to 140 ppm on a dry matter basis, preferentially from 80 ppm to 120 ppm on a dry matter basis, and still more preferably in an amount of about 100 ppm on a dry matter basis. In yet another preferred embodiment, the food composition the rosemary extract is present in an amount ranging from about 55 ppm to about 140 ppm on a dry matter basis. Other available sources for carnosic acid/carnosol can also be selected from Common sage extract (Salvia Oficinalis), from Lamiaceae plant family as thyme, oregano, savero, lemon balm, or hyssop.

In some embodiments of the food according to the present disclosure, the said food can further comprise an amount of carnosic acid and carnosol ranging from about 20 to about 90 ppm on a dry matter basis.

As used herein, an amount of “carnosic acid and carnosol” means the total amount resulting from the sum of (i) the amount of carnosic acid and of (ii) the amount of carnosol in the pet food composition.

Another particular and preferred embodiment of the present disclosure is the inclusion of piperine.

In a preferred aspect, a food composition as disclosed herein further comprises a source of piperine.

In most preferred embodiments of a food composition according to the present disclosure, the said composition can comprise piperine in an amount ranging from 14 to 60 ppm on a dry matter basis.

In a preferred embodiment, the source of piperine consists of a black pepper extract (Piper nigrum). As non-limitative example, it can be mentioned the pepper extract Vetperine® from Sabinsa. Any other sourcing known by the person skilled in the art can also be used. According to such a preferred aspect, a dry dog food composition as described herein further comprises a pepper extract. The said pepper extract can be present in the dry dog food composition in an amount ranging from 15 to 35 ppm as fed, preferentially from 20 to 30 ppm as fed, and still more preferably in an amount of about 27 ppm as fed

In yet another preferred embodiment, a food composition according to the present disclosure can comprise a pepper extract in an amount ranging from about 15 ppm to about 35 ppm. Other available sources for piperine can also be selected from green and white pepper, Genus Piper, or long pepper (Piper longum and Piper officinarum). In yet another preferred embodiment, the food composition according to the present disclosure is characterized in that the turmeric extract is present in the said composition in an amount ranging from about 300 ppm to about 700 ppm and/or the rosemary extract is present in the said composition in an amount ranging from about 50 ppm to about 110 ppm and/or the pepper extract is present in the said composition in an amount ranging from about 15 ppm to about 35 ppm.

The Turmeric extract (rich in curcumin) and the rosemary extract (rich in carnosic acid) are working synergistically to reduce neoplastic cell growth (as shown hereinafter in examples). This extracts combination results in cell apoptosis through Caspase 3/7 activation. Both extracts have antioxidant effects with reducing reactive oxygen species (ROS). In addition turmeric extract and rosemary extract exposure increased activated c-jun N-terminal kinase (JNK). Upon further examination, it has been found that a rosemary treatment causes a significant increase in the cellular accumulation of curcumin. This increase in intracellular curcumin levels can play a role in the synergy. The pepper extract rich in piperine is also able to decrease various tumor cell proliferation (as demonstrated in examples) and in addition it increases the absorption and so bioavailability of a variety of nutrients as curcumin.

In some embodiments, a food composition according to the present disclosure can also comprise a source of polyphenols such as catechins. The source of catechins (flavonoids) can consist preferentially of a green tea extract (Camellia sinensis). As non-limitative example, it can be mentioned the extract from Naturex. Green tea extract refers to an herbal derivative from green tea leaves. Green tea extracts can be created by soft infusions, soft extracts, dry extracts, and partly purified extracts techniques. Green tea extract can comprise green tea catechins (GTC), epigallocatechin (EGC), epicatechin gallate (ECG), epigallocatechin gallate (EGCG) and flavonoids such as kaempferol, quercetin and myricetin. Other common sources of polyphenols are, but not limited to, cacao, Centaurea maculate roots, tea plants including white tea, black tea and Oolonga tea, and grape-wine

In some other embodiments, the food composition according to the present disclosure can also comprise a source of ellagic acid/punicalagin. The source of ellagic acid/punicalagin can consist preferentially of a pomegranate extract (Punica granatum). As non-limitative example, it can be mentioned the extracts from Polinat POE40® or P40P®. Other available source for ellagic acid/punicalagin can be selected from cranberries, walnuts, raspberries, Terminalia catappa tree, Terminalia myriocarpa tree, Combretum molle (velvet bushwillow plant), or Genera Myrtales plant

In a preferred embodiment, the food composition according to the present disclosure further comprises turmeric extract and/or rosemary extract, and/or pepper extract, and/or green tea extract, and/or pomegranate extract.

In some embodiments, a food composition as disclosed herein comprises a combination of a source of curcuminoids and of a rosemary extract. In some of these embodiments, the said pet food composition further comprises a source of piperine.

The present description includes a method for preparing the food composition. The process for the manufacture of the foodstuffs as described can be made according to any method known in the art.

Dry Pet Food Composition

As described elsewhere in the present disclosure, a dry pet food composition according to the present disclosure has a moisture content of less than 15%, such as a moisture content ranging from 1% to 15%, such as a moisture content of about 10%, or such as a moisture content of about 12%.

Thus, the present disclosure encompasses a dry pet food composition comprising from about 10% by weight to about 20% by weight of fat, from about 5% by weight to about 15% by weight of fibers and from about 30% by weight to about 60% by weight proteins, the weight percentages being based on the total weight of dry matter of the composition.

The present disclosure notably relates to a dry dog food composition comprising from about 10% by weight to about 20% by weight of fat, from about 5% by weight to about 15% by weight of fibers and from about 30% by weight to about 50% by weight proteins, the weight percentages being based on the total weight of dry matter of the composition.

In some embodiments, the said dry pet food composition comprises a source of antioxidants comprising Vitamin C and wherein Vitamin C is present in an amount ranging from about 200 ppm to about 400 ppm as fed, and/or Vitamin E in an amount ranging from about 600 ppm to about 1000 ppm as fed and/or carotenoid in an amount ranging from about 2 ppm to about 10 ppm as fed.

In some embodiments, the said dry pet food composition comprises a source of antioxidants comprising Vitamin C, wherein Vitamin C is present in an amount ranging from about 220 ppm to about 440 ppm on a dry matter basis, and/or Vitamin E wherein Vitamin E is present in an amount ranging from about 660 ppm to about 1100 ppm and/or carotenoid wherein carotenoid is present in an amount ranging from about 2 ppm to about 12 ppm, the amounts in ppm being expressed on a dry matter basis.

In some embodiments, the said dry pet food composition comprises a turmeric extract which can be present in the dry dog food composition in an amount ranging from 300 ppm to 700 ppm as fed, preferentially from 400 ppm to 600 ppm as fed, and still more preferably in an amount of 500 ppm as fed.

In some embodiments, the said dry pet food composition comprises a turmeric extract which can be present in the dry dog food composition in an amount ranging from 360 ppm to 780 ppm on a dry matter basis, preferentially from 440 ppm to 670 pm on a dry matter basis, and still more preferably in an amount of 550 ppm on a dry matter basis.

In most preferred embodiments, the said dry pet food composition comprises curcuminoids that are present in an amount ranging from about 250 ppm to about 2000 ppm on a dry matter basis.

In some embodiments, the said dry pet food composition further comprises a source of carnosic acid/carnosol, such as a rosemary extract. The said rosemary extract can be present in the said dry dog food composition in an amount ranging from 50 to 120 ppm as fed, preferentially from 70 to 100 as fed, and still more preferably in an amount of about 90 ppm as fed. In yet another preferred embodiment, the dry pet food composition the rosemary extract is present in an amount ranging from about 50 ppm to about 120 ppm as fed.

In some embodiments, the said dry pet food composition further comprises a source of carnosic acid/carnosol, such as a rosemary extract. The said rosemary extract can be present in the said dry dog food composition in an amount ranging from about 55 to about 130 ppm on a dry matter basis.

In most preferred embodiments of the dry pet food composition, the said composition comprises carnosic acid and carnosol which are present in an amount ranging from 20 ppm to 90 ppm on a dry matter basis.

As used herein, an amount of “carnosic acid and carnosol” means the total amount resulting from the sum of (i) the amount of carnosic acid and of (ii) the amount of carnosol.

In some embodiments, the said dry pet food composition comprises a source of piperine. In some embodiments, the source of piperine consists of a black pepper extract. The said pepper extract can be present in the dry dog food composition in an amount ranging from 15 to 35 ppm as fed, preferentially from 20 to 30 ppm as fed, and still more preferably in an amount of about 27 ppm as fed

In embodiments of the dry pet food composition, the said pepper extract can be present in the pet food composition in an amount ranging from 15 ppm to 35 ppm on a dry matter basis, preferentially from 20 ppm to 30 ppm on a dry matter basis, and still more preferably in an amount of about 30 ppm on a dry matter basis.

In some embodiments of a dry pet food according to the present disclosure, the source of piperine consists of a pepper extract.

In most preferred embodiments, piperine is present in the dry food composition in an amount ranging from 14 ppm to 60 ppm on a dry matter basis.

In yet another preferred embodiment, the dry pet food composition according to the present disclosure is characterized in that the turmeric extract is present in the said composition in an amount ranging from about 300 ppm to about 700 ppm as fed and/or the rosemary extract is present in the said composition in an amount ranging from about 50 ppm to about 120 ppm as fed and/or the pepper extract is present in the said composition in an amount ranging from about 15 ppm to about 35 ppm as fed.

In yet other embodiments, the dry pet food composition according to the present disclosure is characterized in that the turmeric extract is present in the said composition in an amount ranging from about 360 ppm to about 780 ppm on a dry matter basis and/or the rosemary extract is present in the said composition in an amount ranging from about 55 ppm to about 130 ppm and/or the pepper extract is present in the said composition in an amount ranging from about 15 ppm to about 35 ppm on a dry matter basis.

The food composition can be manufactured by mixing together ingredients and kneating in order to make consistent dough that can be cooked. The process of creating a dry pet food is usually done by baking and/or extruding. The dough is typically fed into a machine called an expander and/or extruder, which uses pressurized steam or hot water to cook the ingredients. While inside the extruder, the dough is under extreme pressure and high temperatures. The dough is then pushed through a die (specifically sized and shaped hole) and then cut off using a knife. The puffed dough pieces are made into kibble by passing it through a dryer so that moisture is dropped down to a defined target ensuring stability of the food until consumption. The kibble can then be sprayed with fats, oils, minerals, vitamins, the natural extracts cocktail and optionally sealed into packages. In a preferred embodiment the dry pet food consists of a kibble form. Preferentially, for example and without limitation, kibbles include particulates; pellets; pieces of pet food, dehydrated meat, meat analog, vegetables, and combinations thereof; and pet snacks, such as meat or vegetable jerky, rawhide, and biscuits.

The dry pet food composition is preferably packaged. In this way, the consumer is able to identify, from the packaging, the ingredients in the food product and confirm that it is suitable for the particular pet in question. The packaging can be metal, plastic, paper or card.

Wet Pet Food Composition

In preferred embodiments, the nutritionally complete wet food according to the present disclosure has a moisture content of more than 50%, such as a moisture content ranging from more than 50% to 90%.

Thus, the present disclosure encompasses a wet pet food composition comprising from about 10% by weight to about 20% by weight of fat, from about 5% by weight to about 15% by weight of fibers and from about 30% by weight to about 60% by weight proteins, the weight percentages being based on the total weight of dry matter of the composition.

The present disclosure notably relates to a wet dog food composition comprising from about 10% by weight to about 20% by weight of fat, from about 5% by weight to about 15% by weight of fibers and from about 30% by weight to about 50% by weight proteins, the weight percentages being based on the total weight of dry matter of the composition.

The other ingredients or substances that can be comprised in the said wet pet food, as well as the respective amounts thereof in the composition are already described previously in the general description of a pet food according to the present disclosure.

In some embodiments, the said wet pet food composition comprises a source of antioxidants comprising Vitamin C and wherein Vitamin C is present in an amount ranging from about 200 ppm to about 600 ppm on a dry matter basis, and/or Vitamin E wherein Vitamin E is present in an amount ranging from about 600 ppm to about 2000 ppm on a dry matter basis and/or carotenoid wherein carotenoid is present in an amount ranging from about 30 ppm to about 100 ppm on a dry matter basis.

In some embodiments, the said wet food composition comprises a turmeric extract, as a source of curcuminoids.

Thus, in some embodiments, the said wet food composition comprises a source of curcuminoids.

In some of these embodiments, curcuminoids are present in an amount ranging from about 250 ppm to about 2000 ppm on a dry matter basis.

In some embodiments, the said wet food can further comprise a source of carnosic acid and carnosol. In some of these embodiments, the amount of carnosic acid and carnosol is ranging from about 20 ppm to about 90 ppm on a dry matter basis.

In some embodiments, the said wet food composition comprises a source of piperine. In some of these embodiments, piperine is present in an amount ranging from about 14 ppm to about 60 ppm on a dry matter basis.

In a preferred embodiment, the wet food consists of a chunk form, more particularly of chunks in gravy form. Preferentially, the wet food consists of chunks and gravy, chunks in jelly, loaf, mousse, terrine, bites form.

“Chunks and gravy” products comprise a preformed meat particle prepared by making a meat emulsion and by putting this meat emulsion through a muzzle under pressure and then cooked. A product, such as cooked meat, is diced into chunks, which are eventually mixed with a gravy or sauce. The two components are then filled into a container, usually a can or pouch, which is seamed or sealed and sterilized. As opposed to the ground loaf, chunk and gravy compositions have physically separated, discrete chunks (i.e., pieces of ground meat and grains) as prepared.

These discrete particles are present in the gravy-type liquid in the final container. When serving, chunk and gravy products flow out of the can and can be easily mixed with other dry products.

While the chunk and gravy products allow better integrity of the individual ingredients, the heterogeneous formulation of the chunk and gravy products are sometimes disfavored by consumers.

Wet food compositions are generally packaged in can-like containers and are considered “wet” in appearance because of the moisture contained therein. Two types of wet compositions are generally known in the art. The first is known in the art as “ground loaf.” Loaf products are typically prepared by contacting a mixture of components under heat to produce an essentially homogeneous, intracellular honeycomb-type mass or “ground loaf.” The ground loaf mass is then packaged into a cylindrical container, such as a can. Upon packing, ground loaf assumes the shape of the container such that the ground loaf must be cut when serving to a companion animal.

In a further aspect, the present disclosure provides a process description example for the preparation of a semi-solid moist animal food composition. The process can comprises cutting, chopping, or grinding solid components of the composition with or without aqueous components present. Such solid components can be frozen meats blocks. The components can further be mixed with supplemental components such as nutritional supplements. Typically the mixture is heated to a temperature of from 20 to 70° C. In one arrangement, the mixture is heated to a temperature of around 45° C. The mixing vessel can be suitable for heating via steam injection or heat exchanger or any apparatus which is conventional in the art of food preparation.

The process according to the present disclosure can also be prepared in a batch process.

At the end of the process the composition can be used to fill containers such as cans. The containers are sealed and conventional equipment is used to sterilize the contents. Commercial sterilization is usually accomplished by heating to temperatures of at least 118° C. for an appropriate time depending on the temperature used and the composition. The dry nutritionally complete food composition is preferably packaged. In this way, the consumer is able to identify, from the packaging, the ingredients in the food product and confirm that it is suitable for the particular pet in question. The packaging can be metal, plastic, paper or card.

Semi-Moist Pet Food Composition

As used herein, a “semi-moist” pet food composition has a moisture content ranging from more than 15% to 50%.

Thus, the present disclosure encompasses a semi-moist pet food composition comprising from about 10% by weight to about 20% by weight of fat, from about 5% by weight to about 15% by weight of fibers and from about 30% by weight to about 60% by weight proteins, the weight percentages being based on the total weight of dry matter of the composition.

The present disclosure notably relates to a semi-moist dog food composition comprising from about 10% by weight to about 20% by weight of fat, from about 5% by weight to about 15% by weight of fibers and from about 30% by weight to about 50% by weight proteins, the weight percentages being based on the total weight of dry matter of the composition.

The other ingredients or substances that can be comprised in the said wet pet food, as well as the respective amounts thereof in the composition are already described previously in the general description of a pet food according to the present disclosure.

As conventionally admitted, a semi-moist pet food is the final product of a process allowing obtaining a moisture content value that is intermediate between a dry pet food and a wet pet food.

In some embodiments, the said process can comprise a step of adding a humectant agent. In some embodiments, the said process comprises an extrusion step and a subsequent treatment step with Super-Heated Stream (SHS).

As non-limitative example, semi-moist food can be obtained using Super-Heated Stream (SHS) processes such as processes or methods described in the published patent applications WO2009/018990, WO2009/018996, WO2010/112097, WO2014/122072, WO2016/071372 and/or WO2016/071367.

In a preferred embodiment, the semi-moist food consists of soft semi-moist kibbles.

The food composition is preferably packaged. In this way, the consumer is able to identify, from the packaging, the ingredients in the food product and confirm that it is suitable for the particular pet in question. The packaging can be metal, plastic, paper or card.

The composition as in the form of a pet food product can encompasses any product which a pet consumes in its diet. Thus, the present disclosure covers standard food products as well as pet food snacks (for example, snack bars, biscuits and sweet products). The food product is preferably a cooked product. It can incorporate meat or animal derived material (such as beef, chicken, turkey, lamb, fish, blood plasma, marrow bone etc. or one or more thereof). The product alternatively can be meat free (preferably including a meat substitute such as soya, maize gluten or a soya product) in order to provide a protein source. The product can contain additional protein sources such as soya protein concentrate, milk proteins, gluten etc. The product can also contain a starch source such as one or more grains (e.g. wheat, corn, rice, oats, barley etc.), or can be starch free. The product can include fiber such as chicory, sugar beet pulp, etc. and/or components such as inulin, fructooligosaccharides, probiotics, most preferably, the combined ingredients of the pet food product according to the present disclosure provide all the recommended vitamins and minerals for the particular animal in question (a complete and balanced food) for example as described in National Research Council, 1985, Nutritional Requirements for dogs, National Academy Press, Washington D.C. or Association of America Feed Control Officials, Official Publication 1996.

The food composition described throughout the present disclosure is particularly for use in supporting dogs affected with cancer and undergoing chemotherapy.

By the expression “supporting”, it is commonly understood management of treatment-related issues, minimize sides' effects, keep a good nutritional status, and help fighting cancer, ensure the adequacy of voluntary nutrient intake. Maintain lean body mass, maintain healthy bodyweight, optimal BCS, and prevent malnutrition.

In a preferred embodiment, said cancer consists of lymphoma or mast cells tumors (MCT).

Another particular aspect of the present disclosure consists of a method for supporting a dog affected with a cancer comprising the step of administering to the animal that has a cancer the food composition according to the present disclosure.

The present disclosure also relates to a method for supporting a dog affected with a cancer comprising the step of administering to the animal that has a cancer and that received a chemotherapeutic treatment the food composition according to the present disclosure.

In some embodiments, the present disclosure relates to combined anti-cancer treatments comprising administration of both a chemotherapeutic agent and of a food composition as described herein.

More particularly, the present disclosure concerns a method for the treatment of a cancer comprising the step of administering to a dog that has a cancer a therapeutic effective amount of a chemotherapeutic agent and of the pet food composition according to the present disclosure. In some embodiments, the pet food is a dog food. In some embodiments, the dog food is a dry dog food.

The present disclosure further pertains to a method for the treatment of a cancer in a dog, preferentially lymphoma or mast cell tumor, comprising the step of administering to the animal a therapeutic effective amount of a chemotherapeutic agent in combination with the pet food composition according to the present disclosure. In some embodiments, the pet food is a dog food.

In some embodiments, the dog food is a dry dog food.

The present disclosure also relates to a pharmaceutical composition comprising a chemotherapeutic agent and a pet food composition according to the present disclosure. In some embodiments, the pet food is a dog food. In some embodiments, the dog food is a dry dog food.

According to the present disclosure, a “chemotherapeutic agent” is a chemical agent {e.g., compound or drug) useful in the treatment of cancer, regardless of mechanism of action. Chemotherapeutic agents include compounds used in targeted therapy and conventional chemotherapy. Any such compound or drug known by the person skilled in the art can be used according to the present disclosure.

Preferred chemotherapeutic agent suitable according to the present disclosure are Actinomycin, Bleomycin, Carboplatin, Chlorambucil, Cisplatin, Cyclophosphamide, Cytosine arabinosied, Doxorubicin, L-Asparaginase, Lomustine, Melphalen, Methotrexate, Mitoxantrone, Piroxicam, Prednisone, Vinblastine, Vincristine.

By the expression “therapeutically effective amount”, it must be understood an amount of a chemotherapeutic agent that is sufficient to reduce and/or ameliorate the severity and/or duration of a cancer and/or a symptom related thereto.

The term “combination therapy” or “in combination” means the administration of a therapeutic agent and the dry food composition to treat the therapeutic condition or disorder described in the present disclosure. Such administration encompasses co-administration of these components in a substantially simultaneous manner, or in a sequential manner.

The present disclosure is further illustrated by, without in any way being limited to, the examples below.

### EXAMPLES

### Example 1: Impact of a Novel Diet on Quality of Life in Dogs Receiving Chemotherapy

Objective:

Cancer is one of the most common causes of death for dogs and pet owners are increasingly opting to treat their pets with chemotherapy to prolong a good quality of life (QOL) as long as possible. The purpose of this double-blinded multicenter randomized clinical trial was to investigate the effect of a novel high protein, lower carbohydrate, moderate fat (relative to typical dry foods) diet containing increased fiber content and supplemented in concentrations of omega-3 fatty acids on quality of life (QOL), and gastrointestinal health in dogs undergoing chemotherapy for treatment of mast cell tumors (MCT) and multicentric lymphoma (LSA) within the 8-week feeding period.

A. Materials and Methods

45 chemotherapy-naïve, client-owned dogs newly diagnosed with multicentric LSA or high-grade, metastatic or non-resectable MCT were randomized to receive either a control diet or a test diet for 8 weeks, on top of a standard chemotherapy protocol depending on tumor type (CHOP protocol for LSA; vincristine and/or tyrosine kinase inhibitor or CCNU for MCT). The control diet contained 25% protein, 13% fat, 8.5%, moisture, 1.5% crude fiber. The test diet contained 37% protein, 13% fat, 8.5% moisture, 3.5% crude fiber, 0.6% EPA-DHA, antioxidant supplementation and a different fiber blend than the control diet; both diets were formulated to meet AAFCO nutrient profiles for canine adult maintenance, and had the same energy content.

TABLE 1
Nutritional profiles of control and test diets

| | |
|---|---|
| TEST DIET | CONTROL DIET |

| Nutrient | % | g/Mcal | % | g/Mcal |
|---|---|---|---|---|
| Moisture | 8.5 | 23.01 | 8.5 | 22.34 |
| Protein | 37 | 100.16 | 25 | 65.72 |
| Fat | 13 | 35.19 | 13 | 34.17 |
| Crude Fiber | 3.5 | 9.47 | 1.5 | 3.94 |
| Total dietary fiber | 10 | 27.07 | 6.5 | 17.09 |
| NFE | 31.3 | 84.73 | 45.5 | 119.61 |
| Omega-6 | 2.23 | 6.04 | 2.74 | 7.20 |
| Omega-3 | 1 | 2.71 | 0.13 | 0.34 |
| Omega 6:3 ratio | 2.23 | 2.23 | 21.08 | 21.08 |
| EPA + DHA | 0.6 | 1.62 | 0 | 0.00 |
| L-carnitine (ppm) | 810 | 0.22 | 0 | 0.00 |
| Lutein (ppm) | 5 | 0.00 | 0 | 0.00 |
| Vitamin D (IU/kg) | 1000 | 270.71 | 800 | 210.30 |
| Vitamin E (ppm) | 800 | 0.22 | 100 | 0.03 |
| Vitamin C (ppm) | 300 | 0.08 | | 0.00 |
| Taurine | 0.2 | 0.54 | 0.06 | 0.16 |
| Arginine | 2.75 | 7.44 | 1.48 | 3.89 |
| Energy kcal/kg | 3694 | | 3804 | |

TABLE 2
Guaranteed analysis for control diet

| | |
|---|---|
| MOISTURE (max) | 10% |
| PROTEIN (min) | 23% |
| CRUDE FAT (min) | 10% |
| CRUDE FIBER (max) | 3.5% |
| ASH % (average) | 6.5% |
| NFE % (average) | 45.5% |

Ingredients

brewers rice, chicken by-product meal, oat groats, chicken fat, natural flavors, chicory, wheat gluten, pea fiber, calcium carbonate, potassium chloride, salt, choline chloride, vitamins [DL-alpha tocopherol acetate (source of vitamin E), biotin, D-calcium pantothenate, vitamin A acetate, niacin supplement, pyridoxine hydrochloride (vitamin B6), thiamine mononitrate (vitamin B1), vitamin B12 supplement, riboflavin supplement, folic acid, vitamin D3 supplement], trace minerals [zinc proteinate, zinc oxide, ferrous sulfate, manganese proteinate, manganous oxide, copper sulfate, calcium iodate, sodium selenite, copper proteinate], rosemary extract, preserved with mixed tocopherols and citric acid.

TABLE 3
Guaranteed analysis for Test diet

| | |
|---|---|
| MOISTURE (max) | 10% |
| PROTEIN (min) | 35% |
| CRUDE FAT (min) | 11% |
| CRUDE FIBER (max) | 5.3% |
| ASH % (average) | 6.8% |
| NFE % (average) | 31.4% |

Ingredients

chicken by-product meal, oat groats, brewers rice, wheat gluten, natural flavors, pea fiber, fish oil, chicken fat, chicory, L-arginine, calcium carbonate, potassium chloride, sodium silico aluminate, psyllium seed husk, fructooligosaccharides, salt, hydrolyzed yeast, taurine, N-Butyric acid, L-carnitine, choline chloride, vitamins [DL-alpha tocopherol acetate (source of vitamin E), L-ascorbyl-2-polyphosphate (source of vitamin C), biotin, D-calcium pantothenate, vitamin A acetate, niacin supplement, pyridoxine hydrochloride (vitamin B6), thiamine mononitrate (vitamin B1), vitamin B12 supplement, riboflavin supplement, folic acid, vitamin D3 supplement], trace minerals [zinc proteinate, zinc oxide, ferrous sulfate, manganese proteinate, manganous oxide, copper sulfate, calcium iodate, sodium selenite, copper proteinate], marigold extract (Tagetes erecta L.), rosemary extract, preserved with mixed tocopherols and citric acid.

Dog owners evaluated Quality of Life (QOL), including 12 questions with 5 levels of answer each one, at baseline then every 2 weeks until 8 weeks using a previously published questionnaire designed for dogs with cancer (Iliopoulou M A, Kitchell B E, Yuzbasiyan-Gurkan V. Development of a survey instrument to assess health-related quality of life in small animal cancerCpatients treated with chemotherapy. Am Vet Med Assoc. 2013 Jun. 15; 242(12):1679-87), as well as average fecal score every week until 8 weeks using a 9-point illustrated fecal scoring chart (half points from 1: very liquid stool to 5: very hard stool, 4 being the optimal score).

Body weight, BCS (Body Condition Score on 5-point scale from 1 to 9 with 2 point-intervals, score 5 being optimal) and MCS (Muscle Condition Score: normal muscle mass, mild, moderate or severe muscle loss) were evaluated by veterinary investigators at baseline then every 2 weeks until 8 weeks, CBC (Complete Blood Count) and standard biochemistry were performed on the same schedule.

B. Results

The results of the clinical trials are presented in Table 4 below.

TABLE 4
Demographics data at inclusion (baseline)

| | |
|---|---|
| Control group | Test group |
| n (%) | n (%) |

| | | | | |
|---|---|---|---|---|
| Cancer type | | | | |
| Lymphoma | 16 | (76.19) | 17 | (70.83) |
| MCT | 5 | (23.81) | 7 | (29.17) |
| Patient sex | | | | |
| intact male | 1 | (4.76) | 0 | (0.00) |
| neutered male | 12 | (57.14) | 18 | (75.00) |
| intact female | 0 | (0.00) | 0 | (0.00) |
| spayed female | 8 | (38.10) | 6 | (25.00) |

| | |
|---|---|
| Mean (SD) | Mean (SD) |
| n = 24 | n = 21 |

| | | | | |
|---|---|---|---|---|
| Age | 8.33 | (2.904) | 7.54 | (2.502) |
| Body Weight | 21.02 | (11.334) | 28.53 | (11.267) |
| BCS | 5.29 | (1.347) | 5.13 | (0.612) |

| | | |
|---|---|---|
| Underweight | 1 | 0 |
| Ideal weight | 14 | 20 |
| Overweight | 6 | 4 |

| | | |
|---|---|---|
| MCS | n | n |

| Normal muscle | 20 | | 23 | |
|---|---|---|---|---|
| Mild wasting | 0 | | 1 | |
| Moderate wasting | 0 | | 0 | |
| Severe wasting | 1 | | 0 | |
| | n (%) | | n (%) | |
| Lymphoma | | | | |
| Stage 1 | 1 | (6.25) | 0 | (0.00) |
| Stage 3 | 7 | (43.75) | 11 | (64.71) |
| Stage 4 | 4 | (25.00) | 3 | (17.65) |
| Stage 5 | 4 | (25.00) | 3 | (17.65) |

Lymphoma
Substage

| | | | | |
|---|---|---|---|---|
| A | 13 | (81.25) | 15 | (88.24) |
| B | 3 | (18.75) | 2 | (11.76) |

MCT

| | | | | |
|---|---|---|---|---|
| Grade 2 | 2 | (40.00) | 4 | (57.14) |
| Grade 3 | 2 | (40.00) | 1 | (14.29) |
| Other | 1 | (20.00) | 2 | (28.57) |

14 dogs in the maintenance/control group (10 LSA, 4 MCT) and 22 dogs in the test group (16 LSA, 6 MCT) completed the 8-week study (p=0.061).

B.1. Diet acceptance was good: of the 45 dogs, only 4 (3/21 for control, 1/24 for test, p=0.325) were removed from the study because they would not eat the diet.

Although the number of dogs dropped-off in each group (7/21—33% in control and 2/24—8% in test) was not different (p=0.061), there was a trend for ahigher number of dogs leaving the study m control group compared to test group.

Dogs had stable CBC and biochemistry values over the study.

The results are shown in Table 19 hereafter.

TABLE 5
results of the clinical trial

| | |
|---|---|
| Test diet | |
| Mean (SD) | Reference Range |

Hematocrit %

| Baseline | 42 | (11.58) | * |
|---|---|---|---|
| 4 weeks | 36.15 | (8.45) | |
| 8 weeks | 38.3 | (4.9) | |

White blood cell count
K/uL

| Baseline | 11.65 | (6.09) | * |
|---|---|---|---|
| 4 weeks | 9.49 | (8.32) | |
| 8 weeks | 8.1 | (4.9) | |

Lymphocytes K/uL

| | | |
|---|---|---|
| Baseline | 4.77 | (7.26) |
| 4 weeks | 1.96 | (1.99) |
| 8 weeks | 2.84 | (3.6) |

Albumin g/dL

| | | | | |
|---|---|---|---|---|
| Baseline | 3.14 | (0.41) | 2.7-4.4 | g/dL |
| 4 weeks | 3.66 | (0.39) | | |
| 8 weeks | 3.4 | (0.40) | | |

Aspartate
aminotransferase
(AST)

| | | | | |
|---|---|---|---|---|
| Baseline | 40.96 | (30.49) | 15-66 | U/L |
| 4 weeks | 29.29 | (35.93) | | |
| 8 weeks | 28.45 | (28.04) | | |

Blood urea nitrogen
(BUN)

| | | | | |
|---|---|---|---|---|
| Baseline | 14.75 | (4.27) | 6-31 | mg/dL |
| 4 weeks | 23 | (7.53) | | |
| 8 weeks | 20.64 | (6.23) | | |

Calcium mg/dL

| | | | | |
|---|---|---|---|---|
| Baseline | 9.91 | (1.15) | 8.9-11.4 | mg/dL |
| 4 weeks | 10.12 | (0.63) | | |
| 8 weeks | 9.92 | (0.05) | | |

Lactate mmol/L**

| Baseline | 1.60 | (0.65) | * |
|---|---|---|---|
| 4 weeks | 2.41 | (1.26) | |
| 8 weeks | 1.67 | (0.88) | |

Urine specific gravity

| Baseline | 1.036 | (0.015) | 1.015-1.050 |
|---|---|---|---|
| 4 weeks | 1.031 | (.011) | |
| 8 weeks | 1.038 | (0.017) | |

*The reference ranges for these variables differed somewhat between study sites because these variables were measured in-house. Please see the appendix for the individual ranges for each lab for these variables.
**All values are grouped together here, regardless of methodology of measurement

B.2. Bodyweight and body condition score were stable and in the healthy reference range in both groups throughout the study.

The results are shown in Tables 6 and 7 below.

TABLE 6
Mean body weight for both diet groups at each
time point (dogs with both types of cancer)

| | |
|---|---|
| Control | Test |

| Time point | Mean (SD) | N | Mean (SD) | N |
|---|---|---|---|---|
| Baseline | 21.02 (11.33) | 21 | 28.53 (11.27) | 24 |
| 2 weeks | 19.87 (11.30) | 20 | 26.81 (10.46) | 24 |
| 4 weeks | 20.66 (11.25) | 19 | 26.68 (10.18) | 24 |
| 6 weeks | 19.54 (11.44) | 16 | 27.52 (10.84) | 22 |
| 8 weeks | 21.44 (11.27) | 14 | 27.58 (10.62) | 22 |

TABLE 7
BCS mean and non-adjusted change for both diet groups

| | |
|---|---|
| Control | Test |

| Time point | Mean (SD) | | N | Mean (SD) | | N |
|---|---|---|---|---|---|---|
| Baseline | 5.29 | (1.3) | 21 | 5.13 | (0.6) | 24 |
| 2 weeks | 5.3 | (0.86) | 20 | 4.88 | (0.54) | 24 |
| 4 weeks | 5.32 | (1) | 19 | 5.17 | (0.64) | 24 |
| 6 weeks | 5.44 | (1.09) | 16 | 5 | (0.44) | 22 |
| 8 weeks | 5.21 | (1.12) | 14 | 5 | (0.8) | 22 |

B3. Fecal Scores

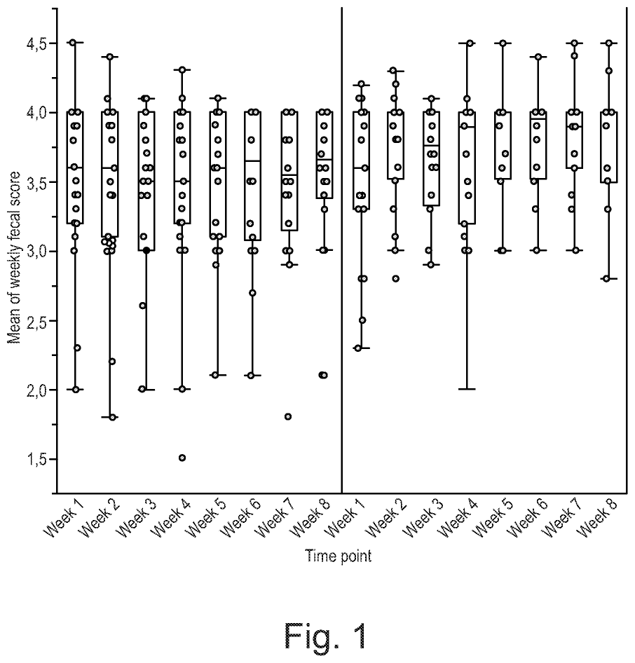

Fetal scores were stable and in the healthy reference range for both groups throughout the study, as it is shown in FIG. 1.

B.4. Quality of Life (QOL) Parameters

From baseline to 8 weeks, 10/12 QOL parameters significantly improved for the dogs on the test diet while only 1/12 improved for dogs eating the control diet.

QOL parameters that showed a significant improvement in test group, and p-values
how much the dog was bothered by the cancer (p=0.003),
how often the dog enjoyed favorite activities (p=0.021),
changes in the dog's sleeping patterns (p=0.009),
the dog's playfulness (p=0.030),
how frequently the dog had signs of illness (p=0.003)
how much the dog enjoyed human interaction (p=0.035)
how often the dog seemed happy (p=0.026)
how often the dog experienced anxiety or fear (p=0.018)
how often the dog experienced mobility problems (p=0.011)
overall QOL for dogs (p=0.010)

Dogs on the test diet also had significant improvement in signs of illness compared to dogs on the control diet (p<0.009).

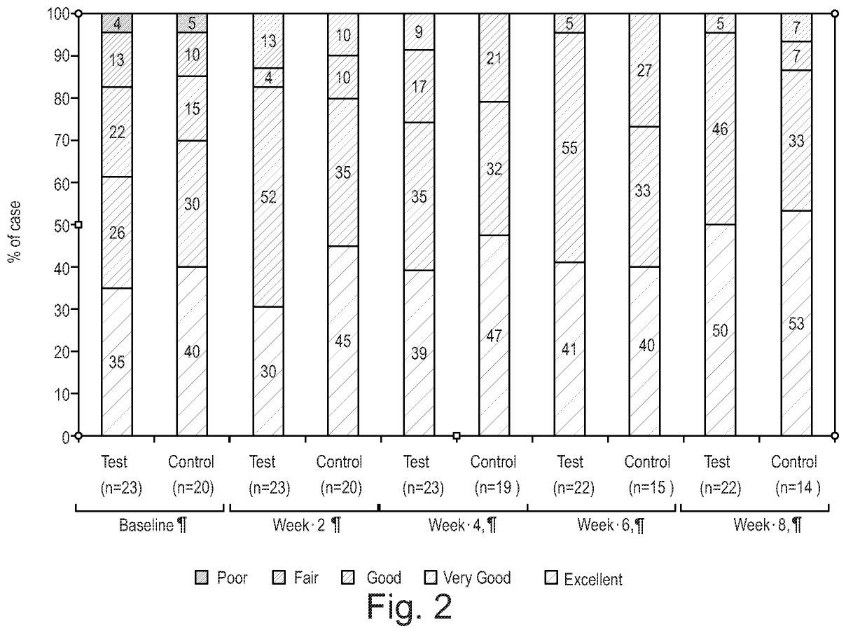

Overall QOL score in each diet group at cancer diagnosis, week 2, 4, 6 and 8 of chemotherapy, as it is shown in FIG. 2, which discloses a scoring of the quality of life of the treated dogs.

Altogether, the results of Example 1 show that the test diet was well tolerated and dogs on test diet showed an improved quality of life after 8 weeks of chemotherapy and diet consumption.

### Example 2: Anti-Proliferative Activities of a Plurality of Plant Extracts

A. Materials and Methods

A.1. Natural extracts were received directly from the manufacturer and the content of each compound of interest based on the manufacturers' purity analysis was verified by a secondary laboratory. Extracts were dissolved at 20 mg/ml, in 100% DMSO to obtain stock solutions before every experiment.

TABLE 8
Characteristics of natural extracts

| Common | | Compound | | | Product | Product |
|---|---|---|---|---|---|---|
| name | Latin name | of interest | Purity^{a} | Manufacturer | name | number |
| Black pepper | Piper | Piperine | 95.02% | Sabinsa | VetPerine ® | FP-0215-06 |
| | nigrum | | | | | |
| Rosemary | Rosmarinus | Carnosic acid | 66.90% | Vitiva | INOLENS70 ® | 302036 |
| | officinalis | | | | | |
| Rosemary | Rosmarinus | Carnosic acid | Min 40% | Vitiva | INOLENS50 | 301999 |
| | officinalis | | | | | |
| Turmeric | Curcuma | Curcuminoids | 87.59% | Naturex | Turmeric | DA251471 |
| | longa | | | | extract | |
| Turmeric | Curcuma | Curcuminoids | 89.41% | Arjuna | BCM-95 ®/Bio- | BCM-05-CG- |
| | longa | | | | Curcumin | 1501-S-48 |
| Pomegranate | Punica | Ellagic acid | 43.6% | Polinat | POE40 ® | POE4011-1711 |
| | granatum | | | | | |
| Pomegranate | Punica | Punicalagins | 35.6% | Polinat | P40P ® | P40P13-2102 |
| | granatum | A/B | | | | |
| Green tea | Camellia | EGCG | 45.76% | Naturex | Green tea | EA140362 |
| | sinensis | Catechins | 83.76% | | extract | |

^{a}Purity value represents the percent of the main compound of interest in each extract as determined by manufacturer (for one specific batch)

A.2. Chemotherapeutic agents used were toceranib phosphate (Palladia™, Zoetis Animal Health) and doxorubicin hydrochloride (Sigma Aldrich). Doxorubicin (positive reference) was prepared at 200 Mm (stock solution for proliferative assay)

A.3. Various canine neoplastic primary cell lines were used representing the different tumor types. The cell lines were grown on tissue culture-treated plates with appropriate medium containing 10% heat inactivated fetal bovine serum and 1% antibiotic-antimycotic. They were grown at 37° C. and 5% CO2 for all experiments and passage of cells.

TABLE 9
canine cancer cell lines

| Cell Line | | |
|---|---|---|
| Name | Cell Type | Origin |
| BR | Mastocytoma | University of California, SF, |
| | | USA, Warren Gold |
| C2 | Mastocytoma | University of California, SF, |
| | | USA, Warren Gold |
| CF33.MT | Tumor mammary | ATCC (#CRL-6227) |
| | gland | |
| CF41.Mg | Tumor mammary | ATCC (#CRL-6232) |
| | gland | |
| CLBL-1 | B-cell lymphoma | University of Veterinary Medicine |
| | | Vienna, Austria, Barbara Rütgen |
| CMT-12 | Mammary gland | Auburn University, Alabama, USA, |
| | carcinoma | R. Curtis Bird |
| D17 | Osteosarcoma | ATCC (#CRL-183) |
| HMPOS | Osteosarcoma | Hokkaido University, Sapporo, Japan |
| K9 | Primary fibrosarcoma | Cornell University, Ithaca, |
| | | USA, Joseph Wakshlag |

All canine cell lines were used in initial screening by ATP-Lite Assay (except CMT-12). Only C2, CMT-12, and D17 cells were used for MTT studies

In addition, canine primary dermal fibroblasts (CDF) were used to investigate effects on normal cells and were propagated and maintained in Prigrow II medium containing 10 HI-FBS and 1% penicillin/streptomycin.

A.4. Various In Vitro Assays to Analyze Cell Proliferation

A.4.1. ATPite™ Proliferation Assay

The different cancer cell lines were plated at the optimal number of cells per well in 90 μL of appropriate medium/well in flat-bottom microtitration 96-well plates. Plates were incubated at 37° C. for 24 hours before treatment in drug free culture medium. After a first dilution process, 10 μl of the test substances (single) were added on plate containing cells 24h after plating. Tumor cell lines were incubated for 72 h at 37° C. under 5% CO2 with 9 concentrations of each natural extract in 1:2 dilution steps. Top dose was 100 μg/ml for each plant extract and 1 μM for doxorubicin. Each concentration was tested in triplicates. Dilutions of each test substance as well as distribution to plates containing cells were performed manually. At least 2 independent experiments were performed for each plant extract. Control cells were treated with vehicles alone. The 5 plant extracts (Green tea leave, pomegranate POE40, rosemary leave INOLENS70, turmeric root and vetperine) were solubilized in DMSO at appropriate concentrations. The 2 selected cancer cell lines (HMPOS and C2) were plated at the optimal number of cells per well in 80 μL of appropriate medium/well in flat-bottom microtitration 96-well plates. Plates were incubated at 37° C. for 24 hours before treatment in drug-free culture medium. After a first dilution process, 10 μl of each test substances was added on plate containing cells. Tumor cell lines were incubated for 72 h at 37° C. under 5% CO2 with 5 concentrations of each test substance alone or in combination. Each condition was done in quadruplicate. Dilutions of each test substance as well as distribution to plates containing cells were performed manually. Two independent experiments were performed. Control cells were treated with vehicle alone (1% DMSO).

At the end of treatments, the cytotoxic activity of tested substances was evaluated by ATP-Lite assay (Ref. 6016949, Perkin-Elmer, and Batch 69-12172). This assay measures the intracellular level of ATP, marker of metabolic activity. This ATP level is quantified by the luminescence emitted by the firefly luciferase, through an ATP-dependent reaction. At the end of the cell treatment, 50 μl of mammalian cell lysis solution were added to the 100 μl of cell suspension per well. The plate was shacked for five minutes in an orbital shaker at 700 rpm. This solution lyses the cells and stabilizes the ATP. Then, 50 μl of substrate solution were added to the wells and the plate was shacked for five minutes in an orbital shaker at 700 rpm. After, the plate was left for ten minutes in the dark and luminescence was measured as LV (Luminescence Value).

Chou-Tallalay CI corresponds to the combination indexes calculated using the appropriate algorithms. Combination of 6 different doses of each extract (1.6, 3.1, 6.3, 12.5 and 25 μg/ml rosemary, 0.8, 1.6, 3.1, 6.2 or 12.5 μg/ml pepper, or turmeric extract). CI values≤0.9 indicate synergism, a CI value>0.9 and <1.1 indicates an additive effect, and CI values≥1.1 indicate antagonism.

A.4.2. MTT Proliferation Assay

Cells were plated at a density of 4×10³ cells per well on 96-well tissue culture-treated flat bottom plates and incubated overnight in complete medium. Cells were treated the following day with DMSO control or extract using a twofold serial dilution for 8 final concentrations ranging from 0.4 to 100 μg mL-1 for 48 h to assess all extracts for potential effectiveness at reducing cellular proliferation. To quantify cellular proliferation, MTT dye assays were performed by adding 30 μL of MTT dye (5 mg mL-1 in phosphate-buffered saline solution) to each well and incubating at 37° C. for 1 h. Media were then aspirated and the cells were solubilized in 200 μL of isopropanol. The optical density of each well was analyzed on a spectrophotometric plate reader (Epoch; Biotek, Winooski, Vt., USA) at a wavelength of 570 nm.

Synergy between extracts was examined using combinations of two extracts at six concentrations: 0.8, 1.7, 3.1, 6.3, 12.5, or 25 μg mL-1. The percent proliferating cells of control for each treatment was pooled from all experiments and is reported as mean f standard error of the mean.

Raw data from proliferation assays (optical density of each well) were normalized to the vehicle alone treatment for individual assays, considered to represent 100% proliferating cells (single or combined treatment). The % proliferating cells was then averaged across each replicate. The IC50 for each extract was then calculated across experiments by Probit analysis. The compound interactions were calculated by multiple drug effect analysis using CalcuSyn software (v.2.11; Biosoft, Cambridge, GB, United Kingdom).

A.4.3. Trypan Blue Exclusion Assay of Cell Viability

The assay was performed on CDF due to the slow rate of proliferation and low metabolic activity of these normal canine cells, precluding productive MTT assays. The effects of extract treatments were compared to the results obtained on the C2, CMT-12, and D17 cell lines. For all cell lines, cells were plated at a density of 5×10³ cells per well and incubated until 60% confluent before treatment with DMSO vehicle control, 6.3 μg mL-1 TE, 6.3 μg mL-1 RE, or a combination of 3.1 μg mL-1 each of TE and RE. After 48 h of treatment, cells were collected and centrifuged. With the exception of the C2 cell line, cells were detached with 0.05% Trypsin/EDTA. The cell pellet was resuspended in 0.1% trypan blue in PBS solution and 1% FBS, loaded on a hemocytometer, and visualized on an inverted microscope. Cells which stained blue were considered non-viable. All values were standardized to the vehicle control treatment which was considered to represent 100% viable cells.

A.5. Soft Agar Growth Assay for Colony Formation

This assay is one of the hallmarks of cell transformation, which is considered the most accurate and stringent in vitro assay for detecting malignant transformation of cells. This clonogenic assay measures proliferation in a semisolid culture media after about 3 weeks by manual counting of colonies. D17 cells were grown in 100 mm cell culture dishes. 6 well culture dishes were set up containing a 0.6% agar type VII solubilized initially at 3% in sterile phosphate buffered saline and reconstituted to 0.6% in complete RPMI media. After 0.5 ml of soft agar is laid as a base layer in the 6 well dishes, each of the cell lines are then suspended at 10,000 cells per ml in 0.6% soft agar in complete RPMI at 38° F. and treatments are applied to the corresponding suspensions immediately before plating in the 6 well dishes. Extracts dosing was chosen based on MTT assay doses that were used in the previous experiments. Typically, cells in soft agar respond to lower doses than what is typically seen in MTT assays. Therefore, doses on the lower end of MTT were chosen (0.4-0.8 ug/ml).

0.5 ml of cells and treatments were plated in triplicate on the same day and allowed to solidify at room temperature in the cell culture hood for 30-60 minutes and places in the 37° F. incubator. Cells were replenished with the appropriate treatments in complete RPMI and 0.6% every 3 days. Colony numbers were counted at 16 days of growth under microscope, by a blinded observer. All data is represented at a percent number of colonies based on DMSO control at 100%.

A.6. Apoptosis-Associated Caspase 3/7 Activation Assay

Cells were plated at a density of 4×10³ cells/well on 96-well tissue culture-treated plates and incubated overnight in complete medium. Cells were treated the following day with DMSO vehicle control, 6.3 μg/mL extract alone, or 3.1 μg/mL each extract in combination for 36 h. Chemotherapeutic drugs at a 50% inhibitory concentration (IC50) were used as a positive control; 12.5 nM toceranib phosphate (Palladia™) was used for the C2 cell line, and 0.3 or 0.5 μM doxorubicin hydrochloride was used for the CMT-12 and D17 cell lines, respectively. Background fluorescence and luminescence was measured in wells containing treatments but no cells. Caspase 3/7 activation was quantified using the ApoLive-Glo™ Multiplex Assay (Promega, Madison, Wis., USA) following manufacturer's instructions. Briefly, after 36 h of treatment, viability reagent was added to the wells and incubated at 37° C. for 30 min and fluorescence was measured at 400Ex/505Em. Next, Caspase-Glo 3/7 Reagent was added to all wells, incubated for 30 min at room temperature, and luminescence was measured. Fluorescence and luminescence was measured using SpectraMax M3 Microplate Reader.

A.7. Flow Cytometry

Cells were plated on 60 mm tissue culture-treated plates (LPS) and incubated in complete medium until 60% confluent. Cells were then treated with medium, DMSO vehicle control, extract alone, or extracts in combination. Cells were treated for 12 h (ROS generation), 24 h (curcumin accumulation), or 48 h (Apoptosis/Necrosis). All flow cytometric analysis was performed on BD FACSCalibur. For all flow cytometry experiments, 10,000 events were collected per sample and then gated based on a forward-scatter/side-scatter plot. The geometric mean fluorescence (GMF) from each treatment was compared to the DMSO treated samples and represented as fold change for all experiments using GMF due to the differences in fluorescence intensity across cell lines.

A.8. Apoptosis and Necrosis Assay

Apoptosis and necrosis was measured after 48 h treatment using Annexin-V and 7-AAD staining. Briefly, cells were detached with Accumax dissociation solutions (Innovative Cell Technologies), collected and centrifuged for 10 min at 500 rcf at 4° C. The cell pellet was washed once with PBS before resuspension in Annexin Binding Buffer (ABB; 10 mM HEPES, 140 mM NaCl, 2.5 mM CaCl2), pH 7.4) at a density of 1×10⁶ cell mL-1. Annexin-V 488 conjugate and 7-Aminoactinomycin D (7-AAD) were added to the cell suspensions and incubated for 15 min at room temperature. ABB was then added to the cell suspension and kept on ice until fluorescence analysis. Events labeled only Annexin-V positive were considered to represent apoptotic cells; events labeled Annexin-V positive and 7-AAD positive were considered to represent necrotic cells.

A.9. Intracellular Reactive Oxygen Species (ROS) Analysis

Since the main constituents of turmeric extract and rosemary extract (curcumin and camosic acid, respectively) have been implicated as antioxidants, Dihydrorhodamine123 (DHR123; Invitrogen) assay was used to determine the amount of ROS present after 12 h treatment with each extract. Briefly, cells were detached using Accumax dissociation Solution, collected and centrifuged for 10 min at 500 ref at 4° C. The pellet was washed once with PBS before resuspension in 1 mL of stain (30 μM DHR123 in DMEM). The cell suspension was then incubated at 37° C. for 30 m, pelleted, and resuspended in 1 mL DMEM and filtered before fluorescence analysis of cells.

A.10. Cellular Accumulation of Curcumin

The cellular accumulation of curcumin was measured by exploiting the auto-fluorescent properties of this compound. After 24 h treatment, cells were detached with Accumax dissociation solution, collected and centrifuged for 10 min at 500 ref at 4° C. The cell pellet was washed once with PBS before resuspension in DMEM, and filtered before fluorescence analysis when excited at a wavelength of 488 nm and then measuring emission using a 530/30 filter.

A.11. Western Blotting (Signaling Pathways)

Cells were plated on 100 mm tissue culture-treated plates (LPS) and incubated overnight in complete medium until 60% confluency was reached. Cells were treated the following day with DMSO vehicle control, 6.3 μg/mL extract alone, or 3.1 μg/mL each extract in combination. Cells were harvested and lysed at 12 h and 24 h after treatment using Mammalian Lysis Buffer (MLB; 25 mM Tris, 100 mM NaCL, 1 mM EDTA, 1% Triton X-100, 0.004% NaF, 1 mM NaVO4, 25 mM-glycerophosphoric acid, 100 □g/ml phenylmethanesulfonyl fluoride, and 1 μg/ml each aprotinin and leupeptin, pH 7.4) and sonication, and then centrifuged for 5 min at 14,000 ref at 4° C. The supernatant was collected and the protein concentration was determined using the Bradford assay (Coomassie-dye). Samples were equilibrated to a common volume (μg/μ) in MLB and 5× laemmili loading buffer. For each protein of interest, 30 μg total proteins were subjected to SDS-PAGE. The proteins were then transferred to 0.45 μm pore size polyvinylidene fluoride membrane for 1 h at 333 mA and then blocked in 5% milk in TBST solution. Membranes were incubated overnight in primary antibody solutions at a dilution of 1:1000 in TBST on a rocking platform at 4° C. Primary antibodies included rabbit stress-activated protein kinase/jun-amino-terminal kinase (SAPK/JNK), and Thr183/Tyr185 phosphorylated-SAPK/JNK (Cell Signaling Technology). Membranes were washed three times with TBST and incubated at room temperature for 1 h in the anti-rabbit IgG horseradish peroxidase-conjugated antibody at a dilution of 1:2000 (Cell Signaling Technology). Membranes were washed three times with TBST and visualized with a chemi-luminescent reagent (Bio-Rad). Digital images were captured using an imaging system (Biospectrum 410). After images were collected, membranes were washed three times in TBST and incubated with a 1:10,000 dilution in TBST of the house-keeping antibody β-Actin (Sigma-Aldrich) for 1 h at room temperature. Membranes were washed, incubated with mouse secondary antibody at a dilution of 1:2000.

B. Results

B.1. Antiproliferative Activity of Single Extract Treatment on Canine Cancer Cell Lines

Anti-Proliferative Activity Determined by ATPlite™ Assay: IC50 Values

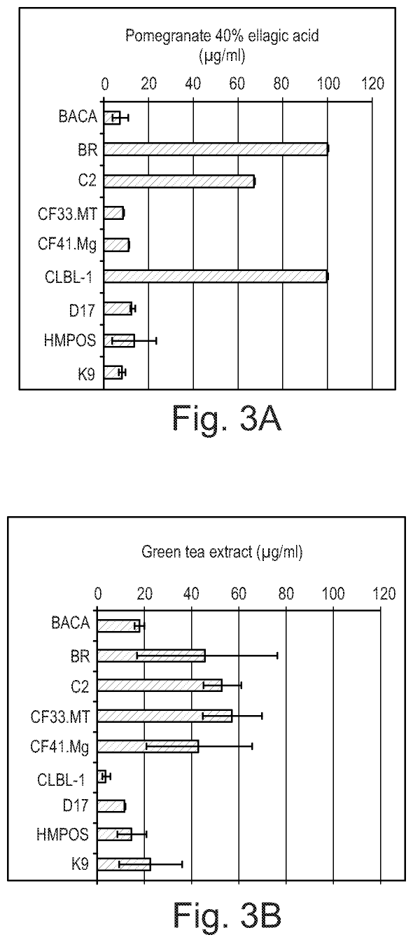

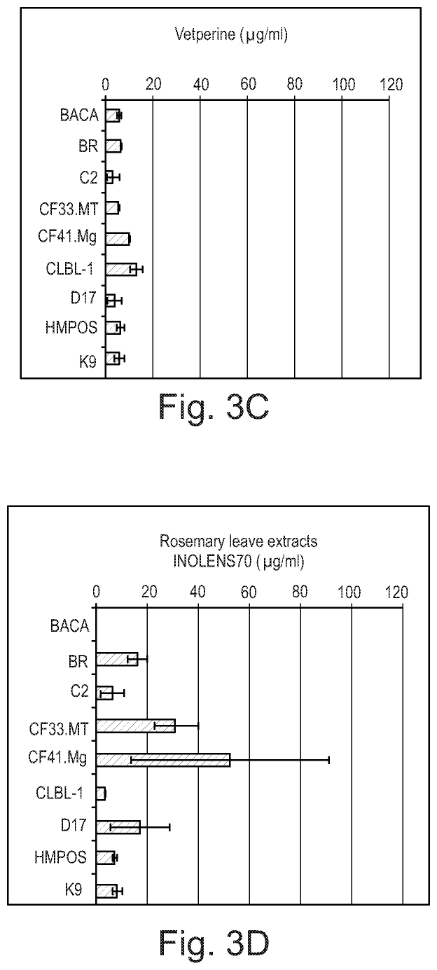

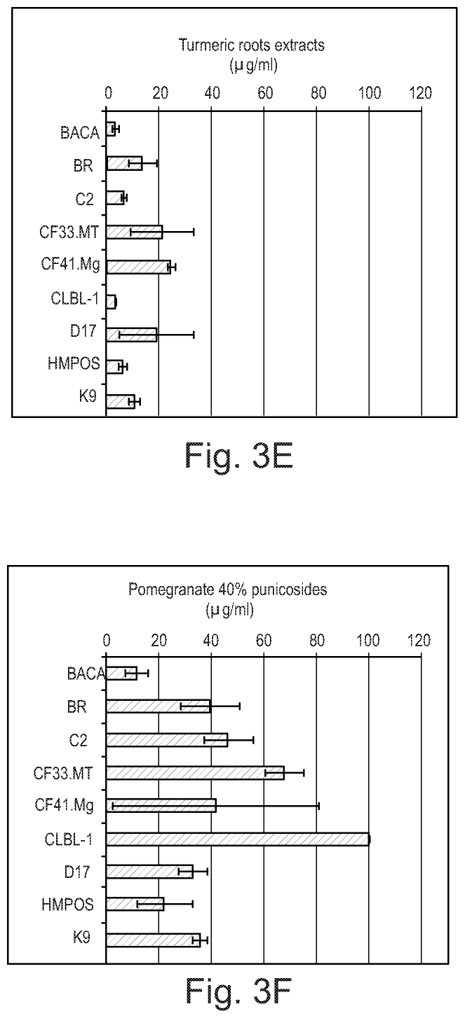

The results of the antiproliferative activity of various plant extracts are depicted in FIGS. 1A to 1F for the extract from promeganate 40% (FIG. 3A), green tea (FIG. 3B), vetperine (FIG. 3C), rosemary leaves (FIG. 3D), turmeric roots (FIG. 3E), promeganate 40% punicosides (FIG. 3F).

Doxorubicin (positive reference): All cell lines were sensitive to Doxorubicin with IC50 values ranging from 11 nM for HMPOS to 375±361 nM for CF41.Mg cell line. Of note CLBL-1 and BACA cell lines were very difficult cell lines to culture, the results regarding these cell lines obtained of the different plant extracts must be cautiously considered.

Anti-proliferative activities have also been assayed according to the MTT assay. The results are presented in Table 10 hereunder.

TABLE 10
Mean IC50 of the extracts with significant anti-
proliferative activity determined by MTT assays

| | Rosemary extract | Tumeric extract | Black pepper | | |
|---|---|---|---|---|---|
| | [70% Camosic Acid] | IC₅₀ | extract | chemotherapy^{a} | |
| | IC₅₀ (μg/mL) | (μg/mL) | IC₅₀ (μg/mL) | IC₅₀ | |
| C2 | 11.9 | 4.8 | 21.8 | 12.5 | nM (6.2 ng mL⁻¹) |
| CMT-12 | 13.0 | 9.1 | 34.5 | 0.3 | μM (163.1 ng mL⁻¹) |
| D17 | 13.6 | 12.3 | 36.5 | 0.5 | μM (271.8 ng mL⁻¹) |

Values were determined by averaging duplicate wells in 4 independent experiments and using Probit analysis. C2 treated with toceranib phosphate, CMT12 and D17 treated with doxorubicin hydrochloride.

B.2. Antiproliferative Activity of Dual Extract Combination Treatment on Canine Cancer Cell Lines

Synergy Results from Two Types of Cancer Cell Lines (C2 and HMPOS) Determined by ATPlite™ Assay

The best synergic combinations were observed with the following compounds at the optimal indicated concentration ranges:
Rosemary (from 0.8 to 6.3 μg/ml)+Pomegranate (from 1.2 to 33 μg/ml)
Rosemary (from 0.8 to 3.1 μg/ml)+Turmeric root (from 0.8 to 3.1 μg/ml)
Pomegranate (from 3.7 to 11 μg/ml)+Turmeric root (from 1.6 to 6.2 μg/ml)

For each combination assay, the synergic effect of compounds was evaluated as the number of synergic Combination Index (CI<0.9) over the number of validated conditions of concentration mixes A condition of concentration mixes was validated in Chou-Tallalay calculation when its combined Fa was within the 0.05 and 0.95 range. A combination of2 extracts was considered as synergic when at least 50% of the validated conditions of concentration mixes shows a CI<0.9.

For all two cell lines, 70% of the synergic effects calculated with additive model method are common to the ones calculated with Chou-Tallalay model. This similarity of results indicates that most of the synergic combinations are validated by two independent calculation methods.

The results are presented in Table 11 hereunder.

B3. Synergy Results from Three Types of Cancer Cell Lines (C2, CMT-12 and D17) Determined by MTT Assay

The combination of turmeric extract and rosemary extract resulted in a most significant decrease in the concentrations of each extract needed to reach an IC50 in all 3 cell lines suggesting a synergistic combination. The results are presented in tables 12, 13 and 14 hereunder.+

TABLE 12
Anti-proliferative activity of the combination of
a rosemary extract and of a turmeric extract on
the proliferation of the C2 cancer cell line.

| | | Turmeric extract Dose (μg/mL⁻¹) | | | | | |
|---|---|---|---|---|---|---|---|
| A | | 0.8 | 1.7 | 3.1 | 6.3 | 12.5 | 25.0 |
| Rosemary extract | 0.8 | 2.551 | 1.786 | 1.307 | 0.362 | 0.128 | 0.216 |
| [70% Carnosic | 1.7 | 2.168 | 0.959 | 0.573 | 0.197 | 0.105 | 0.225 |
| Acid] Dose | 3.1 | 1.339 | 0.678 | 0.373 | 0.152 | 0.118 | 0.208 |
| (μg mL⁻¹) | 6.3 | 0.775 | 0.53 | 0.226 | 0.182 | 0.147 | 0.174 |
| | 12.5 | 0.175 | 0.174 | 0.164 | 0.142 | 0.204 | 0.221 |
| | 25.0 | 0.213 | 0.473 | 0.473 | 0.328 | 0.423 | 0.316 |

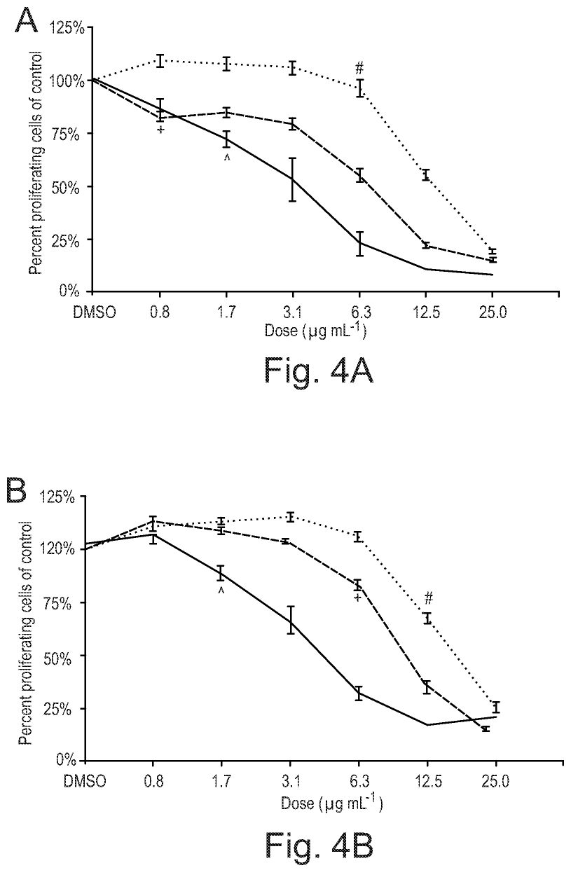

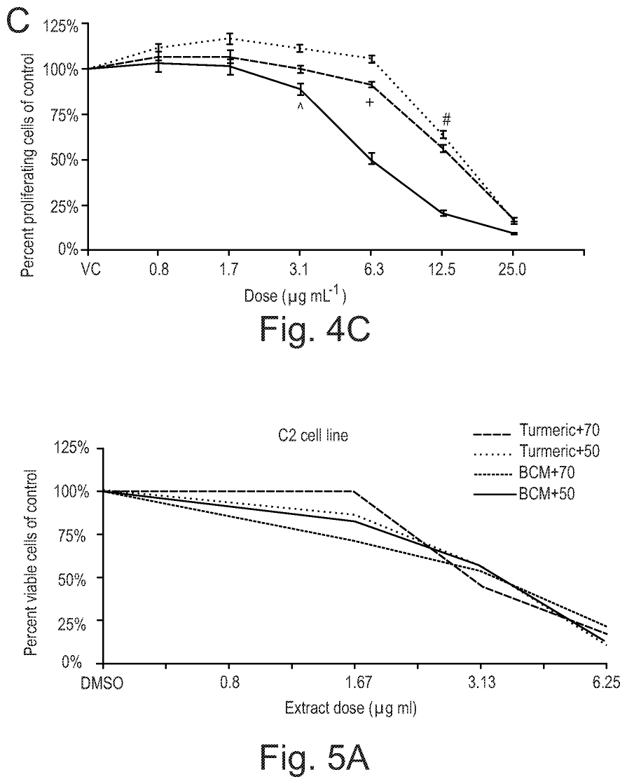

The anti-proliferative activity on the C2 cancer cell line are also depicted in FIG. 4A.

TABLE 13
Anti-proliferative activity of the combination of
a rosemary extract and of a turmeric extract on
the proliferation of the CMT-12 cancer cell line.

| | | Turmeric extract Dose (μg/mL⁻¹) | | | | | |
|---|---|---|---|---|---|---|---|
| B | | 0.8 | 1.7 | 3.1 | 6.3 | 12.5 | 25.0 |
| Rosemary extract | 0.8 | NP | 1.182 | 0.738 | 1.005 | 0.471 | 0.742 |
| [70% Carnosic | 1.7 | 0.611 | 0.682 | 0.655 | 0.793 | 0.355 | 0.703 |
| Acid] Dose | 3.1 | 0.877 | 0.771 | 0.766 | 0.655 | 0.406 | 0.671 |
| (μg mL⁻¹) | 6.3 | 0.971 | 0.859 | 0.789 | 0.647 | 0.524 | 0.775 |
| | 12.5 | 0.629 | 0.603 | 0.523 | 0.528 | 0.687 | 1.048 |
| | 25.0 | 0.853 | 0.944 | 0.871 | 0.869 | 0.882 | 1.601 |

The anti-proliferative activity on the C2 cancer cell line are also depicted in FIG. 4B.

TABLE 14
Anti-proliferative activity of the combination of
a rosemary extract and of a turmeric extract on
the proliferation of the D17 cancer cell line.

| | | Turmeric extract Dose (μg/mL⁻¹) | | | | | |
|---|---|---|---|---|---|---|---|
| C | | 0.8 | 1.7 | 3.1 | 6.3 | 12.5 | 25.0 |
| Rosemary extract | 0.8 | NP | NP | NP | 0.866 | 0.617 | 0.531 |
| [70% Carnosic | 1.7 | NP | NP | NP | 0.909 | 0.59 | 0.527 |
| Acid] | 3.1 | NP | NP | 0.69 | 0.665 | 0.644 | 0.573 |
| | 6.3 | 0.619 | 0.584 | 0.547 | 0.522 | 0.434 | 0.561 |
| | 12.5 | 0.746 | 0.655 | 0.597 | 0.505 | 0.583 | 0.664 |
| | 25.0 | 0.491 | 0.526 | 0.558 | 0.666 | 0.61 | 0.79 |

The anti-proliferative activity on the D17 cancer cell line are also depicted in FIG. 4C. CI values determined by MTT assay for (A) C2, (B) CMT-12, and (C) D17 cell lines when rosemary INOLENS70 extract and turmeric root extract were used in combination at doses ranging from 0.8-25 μg/mL. Values below 0.8 indicate synergy, 0.8-1.2 indicate additive effect, and greater than 1.2 indicate antagonism. NP=Not able to perform calculation.

Lowest dose that induced a significant (p<0.0⁵) decrease in % proliferation compared to DMSO control indicated by +(TE alone), # (RE alone) and \hat{ } (dual extract combination).

B.4. Anti-Proliferative Activity from Different Sourcing of Extracts

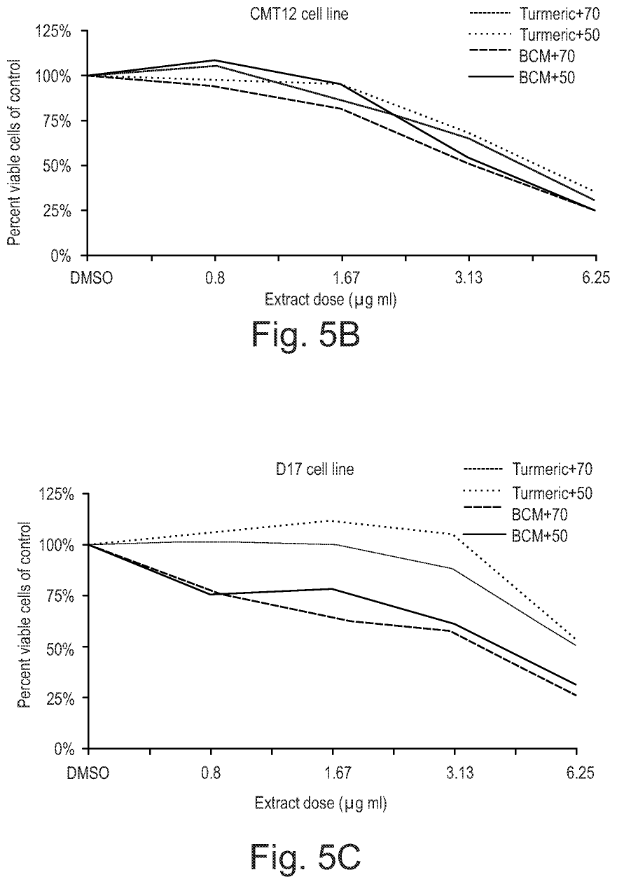

We have also obtained similar anti-proliferative activity of similar extracts using 2 different sourcing of turmeric extract (Naturex or Arjuna) or Rosemary extract (Inolesn70®, Inolens50®). Single treatment or dual combination have been tested on three tumor cell lines (C2, D17, CMT12) and cytoxicity (IC50) has been determined by MTT assays. The results are presented in Table 15 below, as well as in FIGS. 5A, 5B and 5C.

TABLE 15
Anti-proliferative activity of distinct plant extracts on various tumor cell lines

| | | | Rosemary | Rosemary |
|---|---|---|---|---|
| IC50 | Turmeric extract | Turmeric Arjuna | extract | extract |
| Cell | Naturex | BCM-95 | Vitiva | Vitiva |
| lines | (DA251471) | (BCM-05-CG-1501-S-48) | Inolens70 ® | Inolens50 ® |
| C2 | 4.81 μg/mL | 4.04 μg/mL | 11.94 μg/mL | 7.17 μg/mL |
| CMT-12 | 9.09 μg/mL | 9.30 μg/mL | 13.02 μg/mL | 12.59 μg/mL |
| D17 | 12.27 μg/mL | 7.48 μg/mL | 13.55 μg/mL | 15.69 μg/mL |

B.5. Anti-Proliferative Activity of a Turmeric Extract Tested by the Soft Agar Colony Formation Assay

Turmeric extract (curcuminoids) having the largest effect on soft agar with few colonies forming at 27% of control. Rosemary and pepper extracts show mild effects with only 75 and 65% colony formation respectively. The dual curcumin and rosemary treatment showed only 21% formation at 0.4 μg/ml curcumin and 0.8 μg/ml rosemary. The addition of 6.25 μg/ml of pepper extract showed a modest decrease to 12% in the cocktail mixture.

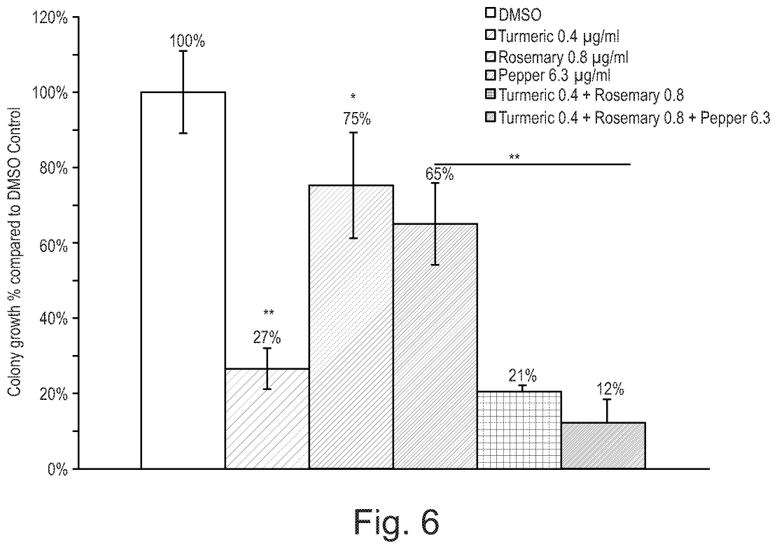

The results are depicted in FIG. 6.

Mean of triplicate, cells were treated every 3 days for 16 days before counting. % mean colony formation as per DMSO vehicle control are reported. * indicates p<0.05, ** indicates p<0.01 when compared to DMSO control.

B.6. Natural Extracts and Chemotherapy Interaction on Growth Inhibition of Cancer Cell Lines

We aimed to examine the interaction between extracts and commonly used chemotherapeutics including palladia/toceranib (tyrosine kinase inhibitor) for mast cell disease (C2 cell line) and doxorubicin (anthracycline antibiotic) for mammary carcinoma and osteosarcoma.

Both turmeric and rosemary extracts (from 0.8 to 1.7 μg/ml) induce mild antagonistic to additive effect, while at 3.1 μg/ml of both extracts there was a definitive additive effect in presence of toceranib phosphate (mastocytoma cell line). When either extract was added at 6.3 μg/ml there was e definitive synergistic effect.

The results are presented in tables 16 to 14 below.

TABLE 16

Turmeric extract
Does(ug mL⁻¹)

| A | | 0.8 | 1.7 | 3.1 | 6.3 |
|---|---|---|---|---|---|
| Rosemary | 0.8 | 1.239 | 1.409 | 1.186 | 0.753 |
| extract | 1.7 | 1.418 | 1.478 | 1.409 | 0.766 |
| Dose | 3.1 | 1.378 | 1.252 | 0.941 | 0.775 |
| (μg mL⁻¹) | 6.3 | 0.946 | 0.842 | 0.736 | 0.707 |

TABLE 17

Turmeric extract
Does(ug mL⁻¹)

| B | | 0.8 | 1.7 | 3.1 | 6.3 |
|---|---|---|---|---|---|
| Rosemary | 0.8 | 1.1 | 1.158 | 0.886 | 0.669 |
| extract | 1.7 | 1.424 | 1.444 | 1.044 | 0.855 |
| Dose | 3.1 | 1.527 | 1.223 | 1.051 | 0.887 |
| (μg mL⁻¹) | 6.3 | 1.018 | 0.887 | 0.808 | 0.729 |

TABLE 18

Turmeric extract
Dose (μg mL⁻¹)

| C | | 0.8 | 1.7 | 3.1 | 6.3 |
|---|---|---|---|---|---|
| Rosemary | 0.8 | 1.104 | 1.182 | 1.11 | 1.016 |
| extract | 1.7 | 1.062 | 1.044 | 0.922 | 1.005 |
| Dose | 3.1 | 1.585 | 1.357 | 1.278 | 1.346 |
| (μg mL⁻¹) | 6.3 | 1.038 | 1.049 | 0.91 | 0.899 |

TABLE 19

Turmeric extract
Dose (μg mL⁻¹)

| D | | 0.8 | 1.7 | 3.1 | 6.3 |
|---|---|---|---|---|---|
| Rosemary | 0.8 | 1.625 | 1.307 | 1.445 | 1.088 |
| extract | 1.7 | 1.622 | 1.272 | 1.002 | 0.895 |
| Dose | 3.1 | 1.46 | 0.89 | 0.836 | 0.809 |
| (μg mL⁻¹) | 6.3 | 1.259 | 0.846 | 0.748 | 0.717 |

TABLE 20

Turmeric extract
Dose (μg mL⁻¹)

| E | | 0.8 | 1.7 | 3.1 | 6.3 |
|---|---|---|---|---|---|
| Rosemary | 0.8 | 2.093 | 1.493 | 1.793 | 1.187 |
| extract | 1.7 | 1.654 | 1.241 | 1.256 | 0.928 |
| Dose | 3.1 | 1.632 | 1.127 | 1.017 | 0.791 |
| (μg mL⁻¹) | 6.3 | 1.462 | 0.918 | 0.894 | 0.718 |

TABLE 21

Turmeric extract
Dose (μg mL⁻¹)

| F | | 0.8 | 1.7 | 3.1 | 6.3 |
|---|---|---|---|---|---|
| Rosemary | 0.8 | 2.111 | 1.764 | 1.38 | 1.048 |
| extract | 1.7 | 1.594 | 1.586 | 1.234 | 0.87 |
| Dose | 3.1 | 1.456 | 1.262 | 0.91 | 0.767 |
| (μg mL⁻¹) | 6.3 | 1.339 | 1.333 | 0.866 | 0.913 |

TABLE 22

Turmeric extract
Dose (μg mL⁻¹)

| G | | 0.8 | 1.7 | 3.1 | 6.3 |
|---|---|---|---|---|---|
| Rosemary | 0.8 | 0.629 | 0.73 | 0.829 | 0.993 |
| extract | 1.7 | 0.685 | 0.743 | 0.84 | 0.923 |
| Dose | 3.1 | 1.032 | 1.035 | 1.028 | 1.053 |
| (μg mL⁻¹) | 6.3 | 1.073 | 1.034 | 1.003 | 0.853 |

TABLE 23

Turmeric extract
Dose (μg mL⁻¹)

| H | | 0.8 | 1.7 | 3.1 | 6.3 |
|---|---|---|---|---|---|
| Rosemary | 0.8 | 1.154 | 1.134 | 1.31 | 1.158 |
| extract | 1.7 | 1.32 | 1.189 | 1.451 | 0.992 |
| Dose | 3.1 | 1.224 | 1.136 | 1.377 | 0.899 |
| (μg mL⁻¹) | 6.3 | 1.083 | 1.081 | 1.17 | 0.69 |

TABLE 24

Turmeric extract
Dose (μg mL⁻¹)

| I | | 0.8 | 1.7 | 3.1 | 6.3 |
|---|---|---|---|---|---|
| Rosemary | 0.8 | 0.361 | 0.399 | 0.421 | 0.554 |
| extract | 1.7 | 0.589 | 0.63 | 0.591 | 0.588 |
| Dose | 3.1 | 0.599 | 0.639 | 0.603 | 0.647 |
| (μg mL⁻¹) | 6.3 | 0.792 | 0.775 | 0.842 | 0.772 |

B.7. Cytotoxic Activity of Extracts Against Cancer Cell Lines without Affecting Normal Cells

Individual extracts at 6.3 μg mL-1 or a combination of 3.1 μg mL-1 TE and 3.1 μg mL-1 RE did not induce a significant decrease in cell viability in the control primary cells

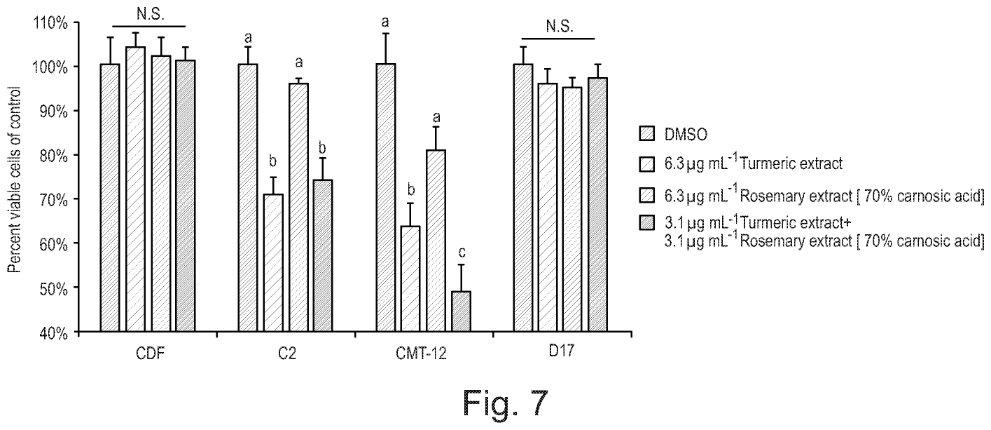

The results are presented in FIG. 7

% viable cells determined by trypan blue exclusion assay are represented as mean+/−SEM in comparison with DMSO vehicle treatment. Within each cell line means not sharing the same letter are significantly different (p<0.05). NS=Not significant.

B.8. Mechanisms by which TE and RE Exert Anti-Proliferative and Cytotoxic Effects Individually and in Combination

Cellular Apoptosis is Induced by Turmeric and Rosemary Extract Treatments

The TE+RE combination treatment resulted in Caspase 3/7 activation and apoptosis in all cell lines, beyond the effects of TE alone.

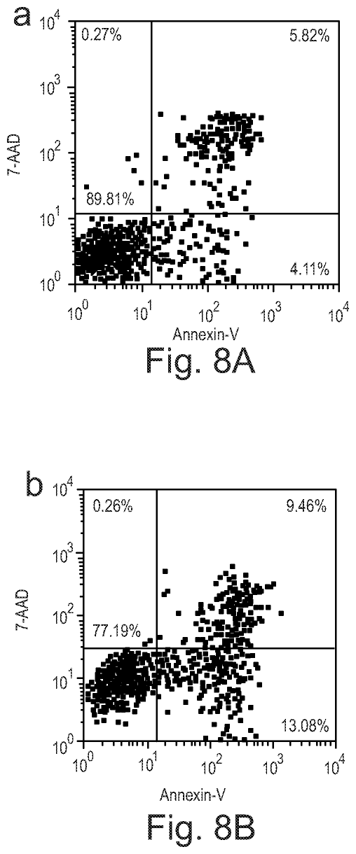

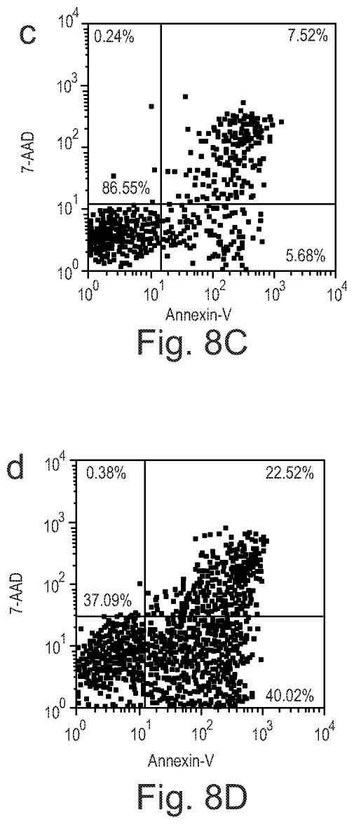

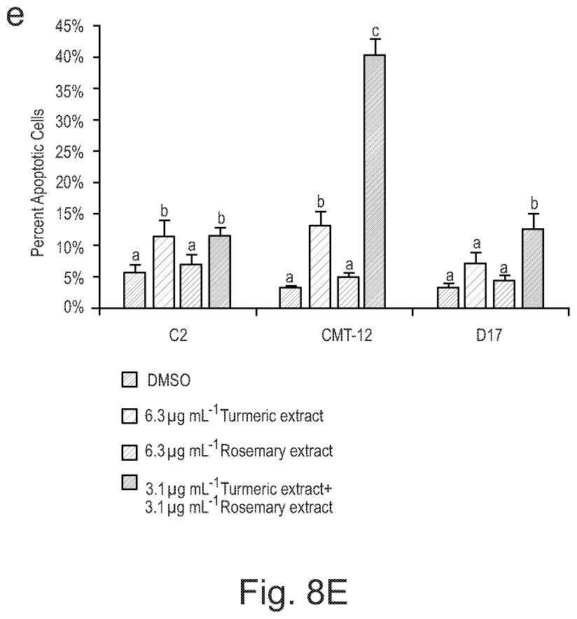

The results are depicted in FIG. 8A to 8E.

FIG. 6 discloses representative quadrant plots of the CMT-12 cell line treated with (FIG. 8A) DMSO, (FIG. 8B) 6.3 μg mL-1 TE, (FIG. 8C) 6.3 μg mL-1 RE, or (FIG. 8D) 3.1 μg mL-1 TE+3.1 μg mL-1 RE are shown. Each quadrant represents the number of events considered live (lower left), early apoptotic (lower right), or late apoptotic/necrotic (upper right). (FIG. 6E) Percent early apoptotic cells (lower right quadrant of Annexin V positive and 7-AAD negative cells) are represented as mean t standard deviation 3 independent replicates). Within each cell line, means with different letters are significantly different from each other (p<0.05)

Apoptosis effect has also been assessed by quantifying the change in caspase 3/7 level per viable cell.

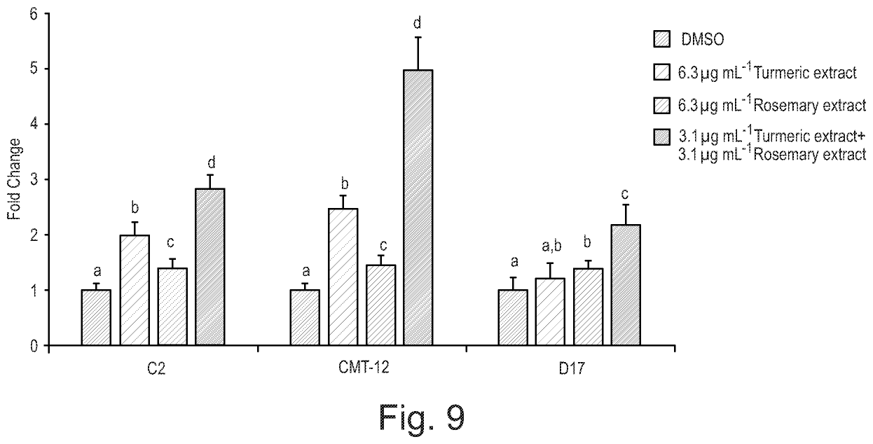

The results are depicted in FIG. 9.

In FIG. 7, activated caspase 3/7 per viable cells was expressed as mean fold change from DMSO control values±standard deviation from 3 independent replicates. Within each cell line, values with different letters are significantly different from each other (C2 p<0.001; CMT-12 p<0.005; D17 p<0.05)

B.9. Antioxidant Activity of TE and RE in Cancer Cell Lines

Both extracts had antioxidant effects with RE reducing reactive oxygen species (ROS) by 40-50% and TE reducing ROS by 80-90%.

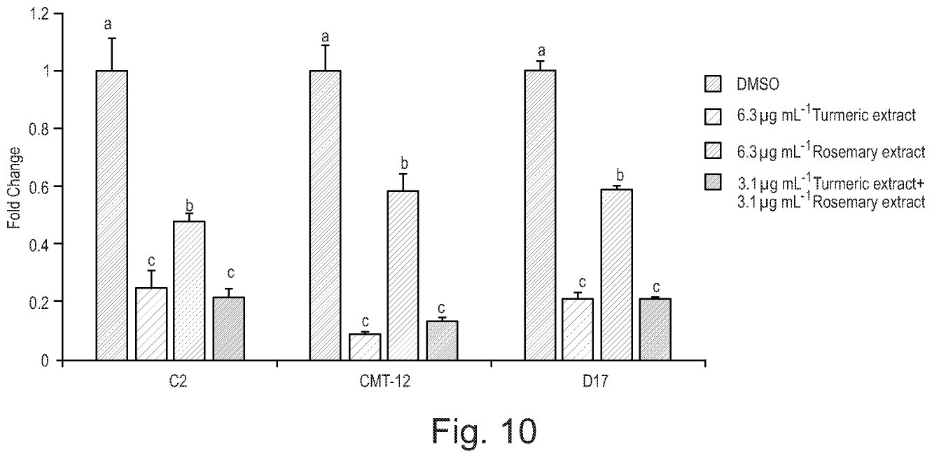

The results are depicted in FIG. 10.

In FIG. 8, values are expressed as mean t standard deviation of four independent replicates.

Reported values are represented as fold change compared to DMSO vehicle control. Within each cell line, means with different letters are significantly different from each other (C2 p<0.05; CMT-12 and D17 p<0.0001)

B.10. TE and RE Promotes SAPK/JNK Cellular Pathway Activation

RE treatment enhanced the c-jun N-terminal kinase (JNK) activity in the C2 cell line and TE+RE exposure increased activated JNK by 4-5 times in the CMT-12 cell line.

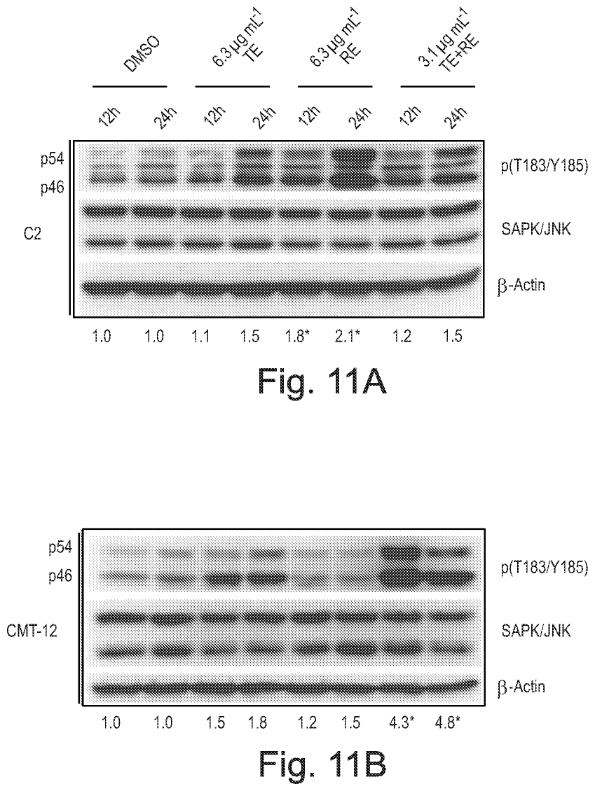

The results are presented in FIGS. 11A (C2 cell line) and 11B (CMT-12 cell line).

In FIGS. 11A and 11B, each blot is a representative of three independent experiments.

Densitometry values represent a ratio of phosphorylated protein to total protein and normalized to DMSO vehicle control of the same time point (mean of 3 separate experiments). Changes in densitometry compared to DMSO control with significance of p<0.05 represented by *. β-Actin was used as a loading control for every blot to ensure even loading of samples.

B.10. Increased Cellular Accumulation of Curcumin Induced by RE Treatment

TE showed that RE treatment caused a significant increase in the cellular accumulation of curcumin by approximately 30% in the C2 and D17 cell lines, and by 4.8-fold in the CMT-12 cell line. This increased curcumin intracellular level can play a role in the synergy exhibited when using TE and RE in combination.

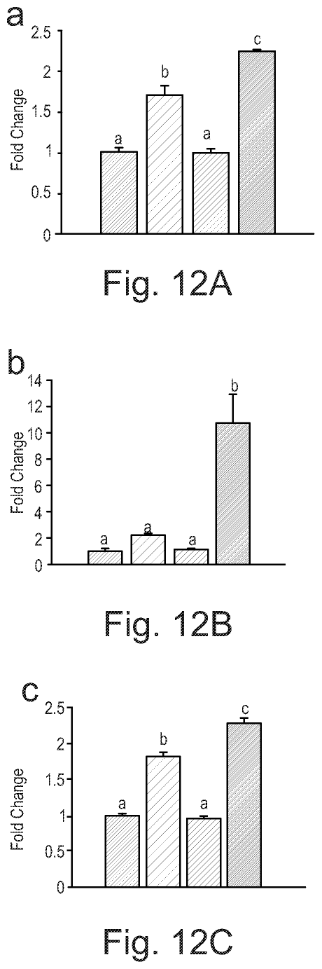

The results are depicted in FIGS. 12A, 12B and 12C.

The C2 (FIG. 12A), CMT-12 (FIG. 12B), and D17 (FIG. 12C) cell lines were treated with the indicated concentration of extracts for 24 h and then cellular accumulation of curcumin was quantified by flow cytometry. Y-axis values represent the fold change in geometric mean fluorescence (GMF) of all cells compared to DMSO control. Reported data are expressed as mean standard deviation of 4 independent replicates. Within each cell line, means with different letters are significantly different from each other (p<0.0001).

### Example 3: In Vivo Example with the Final Composition

The purpose of this prospective multicentric clinical trial is to determine whether the specially formulated experimental diet can improve chemotherapy efficacy with reducing its side effects and maintain quality of life (QoL) of dogs undergoing chemotherapy protocol. The clinical efficacy of this new diet will be evaluated over 1-yr period post cancer diagnosis. The primary objective is to help dogs fighting their cancer, through “support to treatment” maintaining a good nutritional status. The study is designed as double-blind randomized placebo-controlled trial. The primary outcomes are the global health status/QoLscore, GI adverse events and the body weight maintenance. The secondary efficacy parameters will be the other QoL scores (functional and symptom scales), the fecal score (parameter linked to the GI-AEs), median survival, 1-year survival rate, treatment response rates, haematological adverse events (H-AEs), diet assessment by the owner.

Study Design:
Comparative: 2 custom-formulated nutritionally complete and balanced dry diets for dogs with cancer during and after chemotherapy protocol will be compared.
Randomized: Animals will be stratified by study site before being randomly assigned to either the control or test group (utilizing a pre-determined randomization plan).
   2 Groups of dogs (Client-owned) fed for 1-yr period post cancer diagnosis (during and after treatment):
      Group A (n=30)=Test diet
      Ingredients: Dehydrated poultry protein, husked oats, rice, wheat gluten, vegetable fibres, hydrolysed poultry proteins, poultry fats, fish oil, chicory pulp, fatty acid salt, psyllium husks and seeds, marigold extract, exclusive cocktail of natural extracts, minerals, vitamins, antioxidants and preservatives. Guarantee analysis: Moisture (max) 9.5%; protein (min) 36.5%, crude fate (min): 13%; crude fiber (max) 4.8%, ash (average) 7.2%.
      Group B (n=30)=Control diet
      Ingredients: Husked oats, rice, dehydrated poultry protein, poultry fats, wheat gluten, vegetable fibres, hydrolysed poultry proteins, beet pulp minerals, vitamins, antioxidants and preservatives. Guarantee analysis: Moisture (max) 9.5%; protein (min) 25%, crude fate (min): 13%; crude fiber (max) 4.8%, ash (average) 6.8%.

Study Population

Inclusion Criteria
≥1 year of age; Bodyweight≥3 kg
Dogs newly diagnosed nodal diffuse large B-cell lymphoma (DLBCL, centroblastic or immunoblastic) in stages III, IV or V (if limited to bone marrow or peripheral blood involvement)
All animals should be naïve to treatment for the current cancer, but can have been treated for other cancers in the past if greater than 1 year prior.
Owner willing to treat with standard multidrug chemotherapy: CHOP protocol including cyclophosphamide, doxorubicine, vincristine and prednisolone during 25 weeks

The treatment schedule is disclosed in Table 25 hereunder.
Pet owner willing to discontinue all herbal and vitamin supplements. If the dog is receiving any dietary supplements, they can be stopped at the time of enrollment and still be eligible for the study.
Pet owner willing to feed prescribed diet, limit treats to 5% of calories (specific treat recommendations to be provided), and keep monthly QOL and record fecal scores, detailed dietary journal (weekly amount of ingested diet).

Exclusion Criteria
Dogs with neurological troubles induced by lymphoma at inclusion visit
Dogs with lymphoma with other than bone marrow, blood infiltration (as lung, SNC, etc.)
Current diagnosed co-morbidities expected to potentially adversely affect QoL, alter survival time (estimated <3 months), or requiring specific therapeutic diet, for example endocrine diseases, heart disease with ISACHC stage 2-3, IRIS 2-4 chronic kidney disease, etc.
Current antibiotic use within 1 week of study enrollment. The animal could be included if 1 week wash-out before inclusion is performed.
History of gastrointestinal signs (prior to cancer)—chronic vomiting or diarrhea (more than 6 episodes/year or one month of clinical signs) and animal that requires special diet for control (not linked to cancer)
Current prophylactic use of antiemetics and antidiarrheals within 1 week of study enrollment
Use of omega-3 fatty acid supplements (e.g. fish oil, krill oil, flax, borage, evening primrose) within 1 month of study enrollment.
Administration of corticosteroids for the cancer for more than 1 week within the last month (oral route) or of long-action corticosteroids within the last month (whatever the number of injections)

Criteria for Withdrawal/Discontinuation of Participants

Animal will be removed from the study if one of the following criteria is met:
Animal in Partial Response (PR), Stable Disease (SD), or Progressive Disease (PD) after treatment response evaluation (in week 6 of protocol) that requires the modification of the chemotherapy protocol.
Relapse confirmed with cytologic examination (LN FNA) from week 6 (induction phase).
Study diet intolerance—GI signs related to diet within the first 2 weeks of experimental diet introduction that do not resolve within 5 days without medication.
Poor diet compliance, such as: Gap of more than 5 days where the experimental diet is not fed (due to illness, client runs out, tube feeding etc). Excessive use of treats.
Use of additional dietary supplements (except basic glucosamine/chondroitin supplements).

Study Timeline
V1=baseline/inclusion, diagnosis has been done prior to commencement of chemotherapy and diet
V2=after about 1 month of diet (=end of induction phase, in week 6, thus 2 weeks after last doxorubicin injection but before vincristine injection)
V3=after about 3 months of diet (=mid-point maintenance phase, in week 15 before vincristine injection)
V4=after 6 months of diet (=after chemotherapy protocol completion, within the 2 weeks from the last injection, in week 26 or 27)
V5=after 9 months of diet (in between week 39 and 41)
V6=after 1 year of diet (in between week 51 and 53)

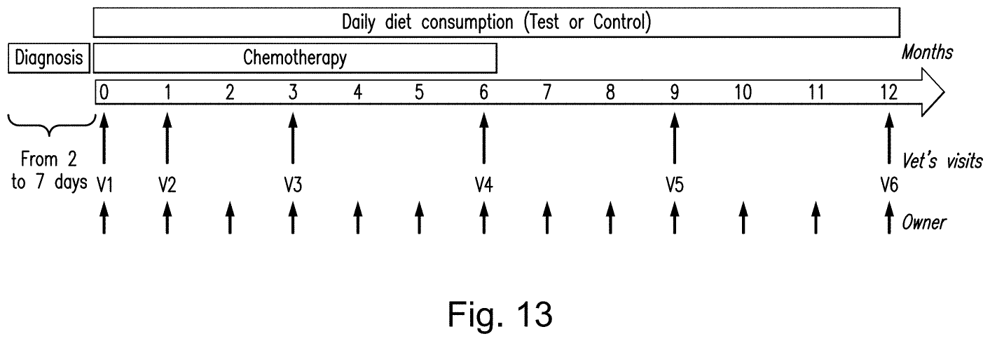

The treatment schedule is further described in FIG. 13.

Parameters
Diagnosis and Clinical staging (V1):
   Complete physical examination
   CBC with blood smear+serum biochemical profile (BUN-CREA-ALT-ALKP-TP-ALB-Ca)+urinalysis (run in-house the same day)
   Cytologic examination and immunophenotyping by flow cytometry on fine-needle aspirates (FNA) of one or two affected lymph nodes (or by immunohistochemistry on biopsy; at the investigator's discretion)
   Thoracic radiographs (2 views: right lateral and Dorso-ventral) (or scanner at the investigator's discretion)
   Abdominal ultrasound examination with imperative splenic/hepatic FNA and cytologic examination
   Bone marrow aspiration and cytology
Body weight; Body Condition Score (BCS 9 pt scale); Muscle Condition Score (MCS WSAVA)
Documenting Medication: chemotherapy protocol (minor changes linked to adaptations rules due to AEs) and other drugs
Documenting adverse events following chemotherapy (grading from 1 to 5 and event date according to the VCOG-CTCAE scale V1.1) only on: the “gastrointestinal” category and the “Blood/Bone Marrow” category
The treatment response evaluation during induction phase (V2):
   Complete response (CR), Partial Response (PR), Stable Disease (SD), and Progressive Disease (PD) performed according to the VCOG consensus: Response evaluation criteria for peripheral nodal lymphoma in dogs v1.0.
   Perform only parameters which were abnormal at the diagnostic visit (only if the animal is in CR)
Standardized Quality of Life questionnaire survey
Fecal scoring (5 pt scale)
Specific Blood biomarkers:
Diet assessment questionnaire
Documenting
   The Recurrence status monitoring up to 12 months (post initial diagnosis) confirmed with LN cytologic examination (FNA). A veterinarian should check the animal following the chemotherapy protocol completion (at least once a month or if evident progressive disease); at the investigator's discretion (allowing to calculate the Progression-free interval)
   Death (natural or euthanasia; likely attributable to cancer or not; unknown origin) allowing to calculate the Median Survival Time.
   Other major events as hospitalization; surgery; injuries, etc.

The treatment protocol is described in Table 26 hereunder.

TABLE 11
Anti-proliferative activity of various combinations of two plant extracts

| | | Green | | Green | | | | Green | Turmeric | | Turmeric |
|---|---|---|---|---|---|---|---|---|---|---|---|
| | | Tea | Rosemary | Tea | Pomegranate | Rosemary | Pomegranate | Tea | Root | Rosemary | Root |
| | | C1 | C2 | C1 | C2 | C1 | C2 | C1 | C2 | C1 | C2 |
| C2 | Nb | 1/12 | 9/18 | 10/22 | 4/20 | 9/18 | 15/21 | 11/19 | 2/16 | 5/16 | 7/17 |
| | C1 < 0.9/Nb | | | | | | | | | | |
| | of validated | | | | | | | | | | |
| | fa | | | | | | | | | | |
| | Median of | 1.34 | 0.78 | 0.97 | 1.43 | 0.84 | 0.53 | 0.84 | 1.87 | 1.00 | 1.14 |
| | validated Fa | | | | | | | | | | |
| HMPOS | Nb | 0/4 | 4/11 | 0/4 | 1/10 | 3/6 | 0/9 | 0/8 | 3/16 | 4/6 | 8/12 |
| | C1 < 0.9/Nb | | | | | | | | | | |
| | of validated | | | | | | | | | | |
| | fa | | | | | | | | | | |
| | Median of | 5.11 | 0.97 | 1.37 | 1.58 | 0.88 | 1.38 | 5.34 | 1.07 | 0.86 | 0.71 |
| | validated Fa | | | | | | | | | | |

| | | Green | | | | Tumeric | | Tumeric | | | |
|---|---|---|---|---|---|---|---|---|---|---|---|
| | | Tea | Vetperine | Rosemary | Vetperine | Root | Vetperine | Root | Pomegranate | Vetperine | Pomegranate |
| | | C1 | C2 | C1 | C2 | C1 | C2 | C1 | C2 | C1 | C2 |
| C2 | Nb | 7/25 | 2/20 | 5/18 | 14/25 | 6/19 | 0/20 | 13/18 | 5/18 | 15/25 | 7/25 |
| | C1 < 0.9/Nb | | | | | | | | | | |
| | of validated | | | | | | | | | | |
| | fa | | | | | | | | | | |
| | Median of | 1.20 | 1.26 | 1.88 | 0.85 | 1.06 | 1.53 | 0.66 | 1.25 | 0.68 | 1.01 |
| | validated Fa | | | | | | | | | | |
| HMPOS | Nb | 0/9 | 1/19 | 1/12 | 0/20 | 12/22 | 8/20 | 8/11 | 6/16 | 6/15 | 1/15 |
| | C1 < 0.9/Nb | | | | | | | | | | |
| | of validated | | | | | | | | | | |
| | fa | | | | | | | | | | |
| | Median of | 2.12 | 1.25 | 1.14 | 1.53 | 0.83 | 1.07 | 0.85 | 0.97 | 0.95 | 1.27 |
| | validated Fa | | | | | | | | | | |

Italic cases correspond to combinations showing synergic values in more than half of the validated Fa.

TABLE 25
treatment schedule

| | | Fre- | | | | | | | | | | | | |
|---|---|---|---|---|---|---|---|---|---|---|---|---|---|---|
| | | quen- | | Week number | | | | | | | | | | |
| Drug | Dose | cy | Route | 1 | 2 | 3 | 4 | 5 | 6 | 7 | 8 | 9 | 10 | 11 |
| Vincris- | 0.7 | once | IV | X | | X | | | X | | X | | | X |
| tine | mg/m² or | a | | | | | | | | | | | | |
| (oncavin) | 0.025 | week | | | | | | | | | | | | |
| | mg/kg | | | | | | | | | | | | | |
| Prednis- | 2 | once | PO | X | | | | | | | | | | |
| olone | mg/kg | daily | | | | | | | | | | | | |
| | or 30 | | | | | | | | | | | | | |
| | mg/m² | | | | | | | | | | | | | |
| | 1/5 | | PO | | X | | | | | | | | | |
| | mg/kg | | | | | | | | | | | | | |
| | or 20 | | | | | | | | | | | | | |
| | mg/m² | | | | | | | | | | | | | |
| | 1 | | PO | | | X | | | | | | | | |
| | mg/kg | | | | | | | | | | | | | |
| | or 10 | | | | | | | | | | | | | |
| | mg/m² | | | | | | | | | | | | | |
| | 0/5 | | PO | | | | X | | | | | | | |
| | mg/kg | | | | | | | | | | | | | |
| | or 5 | | | | | | | | | | | | | |
| | mg/m² | | | | | | | | | | | | | |
| Cyclo- | 250 | once | PO | | X | | | | | X | | | | |
| phosphamide | mg/m² | a | | | | | | | | | | | | |
| | or 10 | week | | | | | | | | | | | | |
| | mg/kg | | | | | | | | | | | | | |
| Doxorubicine | 30 | once | IV | | | | X | | | | | X | | |
| | mg/m² | a | | | | | | | | | | | | |
| | or 1 | week | | | | | | | | | | | | |
| | mg/kg | | | | | | | | | | | | | |

| | Week number | | | | | | | | | | | | | |
|---|---|---|---|---|---|---|---|---|---|---|---|---|---|---|
| Drug | 12 | 13 | 14 | 15 | 16 | 17 | 18 | 19 | 20 | 21 | 22 | 23 | 24 | 25 |
| Vincris- | | | | X | | | | X | | | | X | | |
| tine | | | | | | | | | | | | | | |
| (oncavin) | | | | | | | | | | | | | | |
| Prednis- | | | | | | | | | | | | | | |
| olone | | | | | | | | | | | | | | |
| Cyclo- | | X | | | | | | | | X | | | | |
| phosphamide | | | | | | | | | | | | | | |
| Doxorubicine | | | | | | X | | | | | | | | X |

TABLE 26
treatment protocol

| Visit number | V1 | V2 | V3 | V4* | V5* | V6* |
|---|---|---|---|---|---|---|
| PARAMETERS/ | Screening | ~1-month | ~3-month | 6-month | ~9-month | ~12-month |
| Diet composition | & Baseline | diet | diet | diet | diet | diet |
| Week of chemotherapy | — | 6 | 15 Mid- | 26 End of | — | 1-yr post |
| protocol | | induction | maintenance | chemo | | initial |
| | | phase | | protocol | | diagnosis |
| Visit to the Vet | X | X | X | X | X | X |
| Inclusion/Exclusion criteria | X | | | | | |
| Informed consent criteria | X | | | | | |
| Patient information: Diagnosis and Staging | X | | | | | |
| Information | | | | | | |
| Randomization | X | | | | | |
| Protocol information, provide diet and instructions | X | X | X | X | X | X |
| Fecal scoring: QOL questionnaire; Diet assessment | X | X | X | X | X | X |
| Visit V1: go through the documents with owner | | | | | | |
| without answering questions; Other visits: collect | | | | | | |
| owner paperwork only if data collection not possible | | | | | | |
| using internet and send to CRO | | | | | | |
| Diet history questionnaire | X | | | | | |
| Medication information (Chemotherapy or other) | X | X | X | X | X | X |
| Body weight | X | X | X | X | X | X |
| BCS | X | X | X | X | X | X |
| MCS | X | X | X | X | X | X |
| CBC (including blood smear) ** | X | | | | | |
| Serum Biochemistry panel ** | X | | | X | | X |
| Urine analysis | X | | | | | |
| Serum circulating biomarkers ** (CRP, TK1, HPT, | X | X | | X | | |
| LDH) | | | | | | |
| Blood cooper isotope analysis ** | X | X | X | X | X | X |
| Treatment response evaluation (if lymph nodes | | X | | | | |
| palpation is abnormal, all initial abnormal parameters | | | | | | |
| are performed) | | | | | | |
| Recurrence monitoring | | | X | X | X | X |
| Chemotherapy Adverse Events (VCOG-CTCAE) | | X | X | X | | |

*These visits will be scheduled +/−1 week (try to take place simultaneously with follow-up visit)
** Blood samples obtained as part of routine assessment (before the drug injection)

1. A dog food composition comprising from about 10% by weight to about 20% by weight of fat, from about 5% by weight to about 15% by weight of fibers and from about 30% by weight to about 60% by weight proteins, the weight percentages being based on the total weight of dry matter of the composition.

2. The food composition according to claim 1 which comprises from about 10% by weight to about 20% by weight of fat, from about 5% by weight to about 15% by weight of fibers and from about 30% by weight to about 50% by weight proteins, the weight percentages being based on the total weight of dry matter of the composition.

3. The food composition according to claim 1, which further comprises carbohydrates in an amount ranging from about 15% by weight to about 40% by weight, based on the total weight of dry matter of the composition.

4. The food composition according to claim 1, wherein the food composition is selected from a dry pet food, a semi-moist pet food and a wet pet food.

5. The food composition according to claim 1, which further comprises a source of antioxidants.

6. The food composition according to claim 5, wherein
a) the food composition is a dry pet food composition and the source of antioxidants is selected from Vitamin C, wherein the Vitamin C is present in an amount ranging from about 220 ppm to about 440 ppm on a dry matter basis, Vitamin E, wherein the Vitamin E is present in an amount ranging from about 660 ppm to about 1100 ppm on a dry matter basis, and carotenoid, wherein the carotenoid is present in an amount ranging from about 2 ppm to about 12 ppm on a dry matter basis, or
b) the food composition is a wet pet food composition and the source of antioxidants is selected from Vitamin C, wherein the Vitamin C is present in an amount ranging from about 200 to about 600 ppm on a dry matter basis, Vitamin E, wherein the Vitamin E is present in an amount ranging from about 600 ppm to about 2000 ppm on a dry matter basis, and carotenoid wherein the carotenoid is present in an amount ranging from about 30 ppm to about 100 ppm on a dry matter basis.

7. The food composition according to claim 5, which further comprises a source of curcuminoids.

8. The food composition according to claim 7, wherein the source of curcuminoids is a turmeric extract.

9. The food composition according to claim 7, wherein the food composition is a dry pet food.

10. The food composition according to claim 8, which comprises a turmeric extract in an amount ranging from about 360 ppm to about 780 ppm on a dry matter basis.

11. The food according to claim 9, which comprises curcuminoids in an amount ranging from about 250 to about 2000 ppm on a dry matter basis.

12. The food composition according to claim 9, which further comprises a source of carnosic acid/carnosol.

13. The food composition according to claim 9, which further comprises a rosemary extract.

14. The food composition according to claim 13, wherein the rosemary extract is present in an amount ranging from about 55 ppm to about 130 ppm on a dry matter basis.

15. The food composition according to claim 9, comprising an amount of carnosic acid/carnosol of from about 20 to about 90 ppm on a dry matter basis.

16. The food composition according to claim 9, which further comprises a source of piperine.

17. The food composition according to claim 16 wherein piperine is present in the pet food composition in an amount ranging from 14 to 60 ppm on a dry matter basis.

18. The food according to claim 1, wherein the food is a wet food composition and further comprises a turmeric extract, a source of curcuminoids, carnosic acid/carnosol, piperine, and combinations or mixtures thereof.

19. (canceled)

20. (canceled)

21. The food composition according to claim 18, wherein the curcuminoids is present in an amount ranging from about 250 to about 2000 ppm on a dry matter basis.

22. (canceled)

23. (canceled)

24. (canceled)

25. (canceled)

26. A food composition according to claim 1, for use in supporting a dog affected with cancer and undergoing chemotherapy.
